(19) United States
(12) Patent Application Publication
(10) Pub. No.: US 2025/0129084 A1
(43) Pub. Date: Apr. 24, 2025

(54) Compounds

(71) Applicants: GlaxoSmithKline Intellectual Property Development Limited, Stevenage (GB); University of Dundee, Dundee, Tayside (GB)

(72) Inventors: Robert H Bates, Tres Cantos (ES); LOURDES Encinas, Tres Cantos (ES); Laura Cleghorn, Dundee, Tayside (GB); Simon Green, Dundee, Tayside (GB); Susan H Davis, Dundee, Tayside (GB); Paul Graham Wyatt, Dundee, Tayside (GB)

(21) Appl. No.: 18/839,839

(22) PCT Filed: Feb. 20, 2023
(86) PCT No.: PCT/EP2023/054153

§ 371 Date: Aug. 20, 2024

(30) Foreign Application Priority Data
Feb. 21, 2022 (EP) 22382144.8

(51) Int. Cl. C07D 487/04; A61K 31/519; A61K 45/06; A61P 31/06

(52) U.S. Cl. CPC C07D 487/04; A61K 31/519; A61K 45/06; A61P 31/06

(57) ABSTRACT

The invention relates to compounds or pharmaceutically acceptable salts thereof, compositions containing them, including combinations with at least one additional therapeutic agent, and their use in therapy, for example in the treatment of mycobacterial infections or in the treatment of diseases caused by a mycobacterium, such as tuberculosis.

### FIELD OF THE INVENTION

The invention relates to compounds or pharmaceutically acceptable salts thereof, compositions containing them, including combinations with at least one additional therapeutic agent, and their use in therapy, for example in the treatment of mycobacterial infections or in the treatment of diseases caused by mycobacterium, such as tuberculosis.

### BACKGROUND TO THE INVENTION

Nearly ten million people are infected with tuberculosis (TB) each year, causing an estimated 1.4 million deaths each year, and an additional 0.4 million deaths from TB disease among people living with human immunodeficiency virus (HIV) according to a report published by The World Health Organisation in 2016. If untreated the death rate is more than 50%. Despite available treatments for tuberculosis, the global disease burden remains a major problem owing to Mycobacterium tuberculosis, the causative bacterial agent for TB, becoming resistant to many of the treatments.

In an attempt to prevent resistance to currently available drugs and future approved drugs increasing, TB is treated using combination therapies of three or more drugs. In addition, the treatment of TB often requires therapy using multiple drugs. The standard treatment currently used for drug-susceptible TB is a combination of isoniazid, rifampicin, pyrazinamide and ethambutol, which patients are required to take for two months, followed by isoniazid and rifampicin, only, for a further four months.

Multidrug-resistant TB (MDR-TB) is defined as resistance to at least isoniazid and rifampicin, the two most powerful first-line anti-TB medicines, and extensively drug-resistant TB (XDR-TB) is a form of MDR-TB that is also resistant to at least one fluoroquinolone and any of the second-line anti-TB injectable agents (i.e. amikacin, kanamycin or capreomycin), the two most important classes of medicines in the MDR-TB regimen. For the treatment of M(X)DR TB, it is necessary to administer a regimen of four or more second-line drugs.

The prevalence of TB infection throughout history, is largely due to the ability of Mycobacterium tuberculosis to persist in the host for long periods of time and cause disease even in the face of a highly orchestrated host immune response (Flynn, J. L. & Chan, J. (2001) Annu. Rev. Immunol. 19, 93-129). This unusual ability suggests that mycobacteria may use unique pathogenic mechanisms. Owing to the ever-growing emergence of multi-drug resistant strains of Mycobacterium tuberculosis and continued high incidence of TB, there exists an urgent need to provide further drug compounds for the treatment of TB.

### SUMMARY OF THE INVENTION

The present invention relates to compounds according to Formula (I):

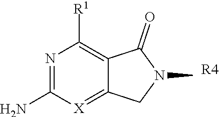

or a pharmaceutically acceptable salt thereof
wherein R⁴ is

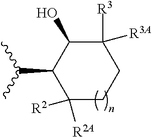

R¹ is methoxy, ethoxy, pyrazolyl, substituted pyrazolyl or —NHMe;
R² and R^{2A} are each independently hydrogen or fluorine;
R³ and R^{3A} are each independently hydrogen or methyl;
X is CH or N;
and n is 1 or 2.

In a second aspect of the invention, there is provided a compound of Formula (I) In a third aspect of the invention, there is provided a pharmaceutically acceptable salt of a compound of Formula (I).

Another aspect of this invention relates to a pharmaceutical composition comprising a compound of Formula (I) or a pharmaceutically acceptable salt thereof; and a pharmaceutically acceptable excipient

Another aspect of this invention relates to a compound of Formula (I) or a pharmaceutically acceptable salt thereof, for use in therapy.

Another aspect of this invention relates to a compound of Formula (I) or a pharmaceutically acceptable salt thereof, for use in the treatment of tuberculosis.

Another aspect of this invention relates to a compound of Formula (I) or a pharmaceutically acceptable salt thereof, for use in the treatment of a mycobacterial infection.

Another aspect of this invention relates to a compound of Formula (I) or a pharmaceutically acceptable salt thereof, for use in the treatment of a disease caused by infection with a mycobacterium.

Another aspect of this invention relates to a method for the treatment of a mycobacterial infection in a human in need thereof. The method comprises administering to the human a therapeutically effective amount of a compound of Formula (I) or a pharmaceutically acceptable salt thereof.

Another aspect of this invention relates to a method for the treatment of a disease caused by infection with a mycobacterium in a human in need thereof, comprising administering to the human a therapeutically effective amount of a compound of Formula (I) or a pharmaceutically acceptable salt thereof.

Another aspect of this invention relates to a use of a compound of Formula (I) or a pharmaceutically acceptable salt thereof, in the manufacture of a medicament for use in the treatment of a mycobacterial infection or a disease caused by infection with a mycobacterium.

Another aspect of this invention relates to a combination of (a) a compound of Formula (I) or a pharmaceutically acceptable salt thereof; and (b) at least one other anti-mycobacterial agent.

Another aspect of this invention relates to a kit comprising a compound of Formula (I) or a pharmaceutically acceptable salt thereof and instructions for administering to a human in need thereof.

### DETAILED DESCRIPTION OF THE INVENTION

### Terms and Definitions

It will be understood that a phrase such as “a compound of Formula (I) or a pharmaceutically acceptable salt thereof” is intended to encompass the compound of Formula (I), a pharmaceutically acceptable salt or solvate of the compound of Formula (I), or any pharmaceutically acceptable combination of these. Thus by way of non-limiting example used here for illustrative purpose, “a compound of Formula (I) or a pharmaceutically acceptable salt thereof” encompasses a pharmaceutically acceptable salt of a compound of Formula (I) which is present as a solvate or a hydrate, and this phrase also encompasses a mixture of a compound of Formula (I) and a pharmaceutically acceptable salt of a compound of Formula (I).

It is to be further understood that references herein refer to a compound of Formula (I) or a pharmaceutically acceptable salt thereof includes a compound of Formula (I) as a free base or as a pharmaceutically acceptable salt thereof.

The term “pharmaceutically acceptable” refers to those compounds (including salts), materials, compositions, and dosage forms which are, within the scope of sound medical judgment, suitable for use in contact with the tissues of human beings and animals without excessive toxicity, irritation, or other problem or complication, commensurate with a reasonable benefit/risk ratio.

As used herein, the term “pharmaceutically acceptable salts” represents salts that retain the desired biological activity of the human compound and exhibit minimal undesired toxicological effects. These pharmaceutically acceptable salts may be prepared in situ during the final isolation and purification of the compound, or by separately reacting the purified compound in its free acid or free base form with a suitable base or acid, respectively.

Pharmaceutically acceptable salts include, amongst others, those described in Berge, J. Pharm. Sci., 1977, 66, 1-19, or those listed in P H Stahl and C G Wermuth, editors, Handbook of Pharmaceutical Salts; Properties, Selection and Use, Second Edition Stahl/Wermuth: Wiley-VCH/VHCA, 2011 (see http://www.wiley.com/WileyCDA/WileyTitle/productCd-3906390519.html).

Where the compound functionality allows, suitable pharmaceutically acceptable salts of the compound of Formula (I) can be formed, which include acid addition salts. Acid addition salts may be formed by reaction with the appropriate acid, optionally in a suitable solvent such as an organic solvent, to give the salt which can be isolated by crystallisation and filtration. Representative pharmaceutically acceptable acid addition salts include, but are not limited to, 4-acetamidobenzoate, acetate, adipate, alginate, ascorbate, aspartate, benzenesulfonate (besylate), benzoate, bisulfate, bitartrate, butyrate, calcium edetate, camphorate, camphorsulfonate (camsylate), caprate (decanoate), caproate (hexanoate), caprylate (octanoate), cinnamate, citrate, cyclamate, digluconate, 2,5-dihydroxybenzoate, disuccinate, dodecylsulfate (estolate), edetate (ethylenediaminetetraacetate), estolate (lauryl sulfate), ethane-1,2-disulfonate (edisylate), ethanesulfonate (esylate), formate, fumarate, galactarate (mucate), gentisate (2,5-dihydroxybenzoate), glucoheptonate (gluceptate), gluconate, glucuronate, glutamate, glutarate, glycerophosphorate, glycolate, hexylresorcinate, hippurate, hydrabamine (N,N′-di(dehydroabietyl)-ethylenediamine), hydrobromide, hydrochloride, hydroiodide, hydroxynaphthoate, isobutyrate, lactate, lactobionate, laurate, malate, maleate, malonate, mandelate, methanesulfonate (mesylate), methylsulfate, mucate, naphthalene-1,5-disulfonate (napadisylate), naphthalene-2-sulfonate (napsylate), nicotinate, nitrate, oleate, palmitate, p-aminobenzenesulfonate, p-aminosalicyclate, pamoate (embonate), pantothenate, pectinate, persulfate, phenylacetate, phenylethylbarbiturate, phosphate, polygalacturonate, propionate, p-toluenesulfonate (tosylate), pyroglutamate, pyruvate, salicylate, sebacate, stearate, subacetate, succinate, sulfamate, sulfate, tannate, tartrate, teoclate (8-chlorotheophyllinate), thiocyanate, triethiodide, undecanoate, undecylenate, and valerate.

As used herein, the term “therapeutically effective amount” means any amount which, as compared to a corresponding human who has not received such amount, results in improved treatment, healing, prevention, or amelioration of a disease, disorder, or side effect, or a decrease in the rate of advancement of a disease or disorder. An appropriate “therapeutically effective amount” will depend upon a number of factors including, for example, the age and weight of the human, the precise condition requiring treatment and its severity, the nature of the formulation, and the route of administration, and will ultimately be at the discretion of the attendant physician.

As used herein, “treatment” and “treating”, are used interchangeably herein, and refer to an approach for obtaining beneficial or desired results including, but not limited to, therapeutic benefit. By therapeutic benefit in meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient can still be afflicted with the underlying disorder. The term “treat’, in all its verb forms, is used herein to mean to relieve, alleviate, prevent, and/or manage at least one symptom of a disorder in a human.

As used herein, the term “solvate” refers to forms of the compound of Formula (I), or a pharmaceutically acceptable salt thereof, that are associated with a solvent, usually by a solvolysis reaction. This physical association may include hydrogen bonding. Conventional solvents include water, methanol, ethanol, acetic acid, DMSO, THF, diethyl ether, and the like. The compounds described herein may be prepared, e.g., in crystalline form, and may be solvated. Suitable solvates include pharmaceutically acceptable solvates and further include both stoichiometric solvates and non-stoichiometric solvates. In certain instances, the solvate will be capable of isolation, for example, when one or more solvent molecules are incorporated in the crystal lattice of a crystalline solid. “Solvate” encompasses both solution-phase and isolatable solvates. Representative solvates include hydrates, ethanolates, and methanolates. In some embodiments, the compounds of Formula (I) or pharmaceutically acceptable salts thereof may be isolated, formed, and/or administered as a solvate.

As used herein, the term “hydrate” refers to forms of the compound of Formula (I), or a pharmaceutically acceptable salt thereof, that are associated with water. Typically, the number of the water molecules contained in a hydrate of a compound is in a definite ratio to the number of the compound molecules in the hydrate. Therefore, a hydrate of a compound may be represented, for example, by the general formula R•x H₂O, wherein R is the compound, and x is a number greater than 0. A given compound may form more than one type of hydrate, including, e.g., monohydrates (x is 1), lower hydrates (x is a number greater than 0 and smaller than 1, e.g., hemihydrates (R•0.5 H₂O)), and polyhydrates (x is a number greater than 1, e.g., dihydrates (R•2 H₂O) and hexahydrates (R•6 H₂O)). In some embodiments, the compounds of Formula (I) or pharmaceutically acceptable salts thereof may be isolated, formed, and/or administered as a hydrate.

It is also to be understood that compounds that have the same molecular formula but differ in the nature or sequence of bonding of their atoms or the arrangement of their atoms in space are termed “isomers”. Isomers that differ in the arrangement of their atoms in space are termed “stereoisomers”. Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory {i.e., as (+) or (−)-isomers respectively). A chiral compound can exist as either individual enantiomer or as a mixture thereof. A mixture containing equal proportions of the enantiomers is called a “racemic mixture”. In some embodiments, the compounds of Formula (I) or pharmaceutically acceptable salts thereof, can contain one or more asymmetric centres (also referred to as a chiral centres) and may, therefore, exist as individual enantiomers, diastereoisomers, or other stereoisomeric forms, or as mixtures thereof. Reference herein to compounds of Formula I or pharmaceutically acceptable salts thereof refer to any single or mixture of stereoisomers unless otherwise designated.

The term “polymorph” refers to a crystalline form of a compound (or a salt, hydrate, or solvate thereof). All polymorphs have the same elemental composition. Different crystalline forms usually have different X-ray diffraction patterns, infrared spectra, melting points, density, hardness, crystal shape, optical and electrical properties, stability, and solubility. Recrystallization solvent, rate of crystallization, storage temperature, and other factors may cause one crystal form to dominate. Various polymorphs of a compound can be prepared by crystallization under different conditions. Polymorphic forms of a compound of Formula (I) or a pharmaceutically acceptable salt thereof may be characterised and differentiated using a number of conventional analytical techniques, including, but not limited to, X-ray powder diffraction (XRPD), infrared spectroscopy (IR), Raman spectroscopy, differential scanning calorimetry (DSC), thermogravimetric analysis (TGA) and solid-state nuclear magnetic resonance (ssNMR).

### Compounds

This invention relates to compounds of Formula (I):

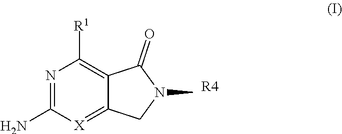

wherein R⁴ is

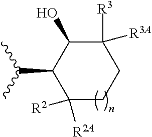

or pharmaceutically acceptable salts thereof.

In some embodiments, R¹ is methoxy, ethoxy, pyrazolyl, substituted pyrazolyl or —NHMe. In some embodiments, R¹ is methoxy. In other embodiments, R¹ is ethoxy. In still other embodiments, R¹ is —NHMe. In yet other embodiments, R¹ is pyrazolyl. In some embodiments, R¹ is 1H-pyrazol-1-yl. In yet further embodiments, R¹ is a substituted pyrazolyl, such as 1H-pyrazol-1-yl substituted at one or more of the 3, 4 and 5 positions on the ring. In some embodiments, R¹ is 1H-pyrazol-1-yl substituted with an R⁵ group at the 4 position (as shown below), wherein R⁵ is selected from OMe, OH, Me, F, OCH₂cPr, OEt, OCH₂CF₃.

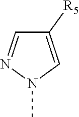

In some embodiments, R¹ is 4-ethoxy-1H-pyrazol-1-yl.

In some embodiments, R² and R^{2A} are each independently selected from H or fluorine. In some embodiments, R² and R^{2A} are each fluorine. In other embodiments, R² and R^{2A} are each hydrogen (i.e., H). In still other embodiments, R² is hydrogen and R^{2A} is fluorine or R² is fluorine and R^{2A} is hydrogen, where R² and R^{2A} can be in either a R- or S-stereochemical configuration.

In some embodiments, R³ and R^{3A} are each independently selected from hydrogen or methyl. In some embodiments, R³ and R^{3A} are each methyl. In other embodiments, R³ and R^{3A} are each hydrogen. In still other embodiments, R³ is hydrogen and R^{3A} is methyl or R³ is methyl and R^{3A} is hydrogen, where R³ and R^{3A} can be in either a R- or S-stereochemical configuration.

In some embodiments; X is CH or N. In some embodiments, X is CH. In other embodiments, X is N.

In some embodiments, n is 1 or 2. In some embodiments, n is 1 to form a cyclohexyl ring. In other embodiments, n is 2 to form a cycloheptyl ring.

In some embodiments, the compounds of Formula (I) are depicted in Table 1.

| Compound | | |
|---|---|---|
| No. | Structure | Chemical Name |
| 45 | 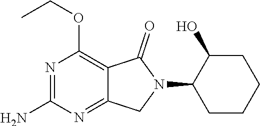 [SMILES: CCOc1nc(N)nc2c1C(=O)N([C@@H]1CCCC[C@@H]1O)C2] | 2-Amino-4-ethoxy-6-[(1R,2S)-2- hydroxycyclohexyl]-7H-pyrrolo[3,4- d]pyrimidin-5-one |
| 46 | 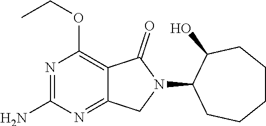 [SMILES: CCOc1nc(N)nc2c1C(=O)N([C@@H]1CCCCC[C@@H]1O)C2] | 2-Amino-4-ethoxy-6-((1R,2S)-2- hydroxycycloheptyl)-6,7-dihydro-5H- pyrrolo[3,4-d]pyrimidin-5-one |
| 47 | 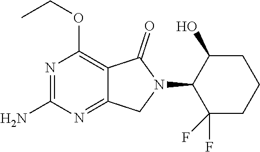 [SMILES: CCOc1nc(N)nc2c1C(=O)N([C@H]1[C@@H](O)CCCC1(F)F)C2] | 2-Amino-6-((1S,6S)-2,2-difluoro-6- hydroxycyclohexyl)-4-ethoxy-6,7- dihydro-5H-pyrrolo[3,4-d]pyrimidin-5- one |
| 48 | 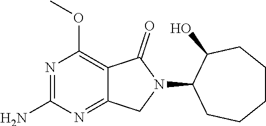 [SMILES: COc1nc(N)nc2c1C(=O)N([C@@H]1CCCCC[C@@H]1O)C2] | 2-Amino-6-((1R,2S)-2- hydroxycycloheptyl)-4-methoxy-6,7- dihydro-5H-pyrrolo[3,4-d]pyrimidin-5- one |
| 49 | 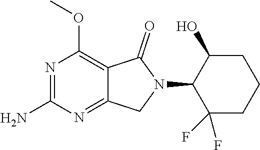 [SMILES: COc1nc(N)nc2c1C(=O)N([C@H]1[C@@H](O)CCCC1(F)F)C2] | 2-Amino-6-((1S,6S)-2,2-difluoro-6- hydroxycyclohexyl)-4-methoxy-6,7- dihydro-5H-pyrrolo[3,4-d]pyrimidin-5- one |
| 50 | 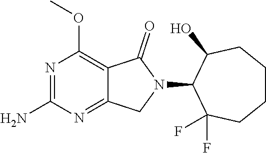 [SMILES: COc1nc(N)nc2c1C(=O)N([C@H]1[C@@H](O)CCCCC1(F)F)C2] | 2-Amino-6-((1S,7S)-2,2-difluoro-7- hydroxycycloheptyl)-4-methoxy-6,7- dihydro-5H-pyrrolo[3,4-d]pyrimidin-5- one |
| 51 | 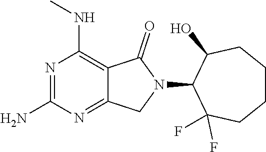 [SMILES: CNc1nc(N)nc2c1C(=O)N([C@H]1[C@@H](O)CCCCC1(F)F)C2] | 2-Amino-6-((1S,7S)-2,2-difluoro-7- hydroxycycloheptyl)-4-(methylamino)- 6,7-dihydro-5H-pyrrolo[3,4-d]pyrimidin- 5-one |
| 52 | 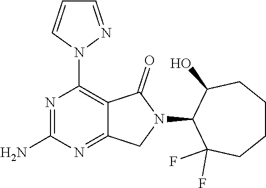 [SMILES: Nc1nc2c(c(-n3cccn3)n1)C(=O)N([C@H]1[C@@H](O)CCCCC1(F)F)C2] | 2-Amino-6-((1S,7S)-2,2-difluoro-7- hydroxycycloheptyl)-4-(1H-pyrazol-1- yl)-6,7-dihydro-5H-pyrrolo[3,4- d]pyrimidin-5-one |
| 53 | 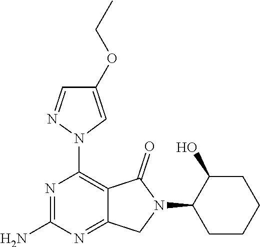 [SMILES: CCOc1cnn(-c2nc(N)nc3c2C(=O)N([C@@H]2CCCC[C@@H]2O)C3)c1] | 2-amino-4-(4-ethoxy-1H-pyrazol-1-yl)-6- ((1R,2S)-2-hydroxycyclohexyl)-6,7- dihydro-5H-pyrrolo[3,4-d]pyrimidin-5- one |
| 54 | 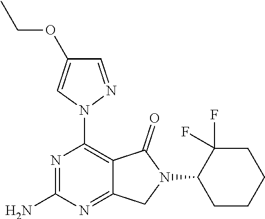 [SMILES: CCOc1cnn(-c2nc(N)nc3c2C(=O)N([C@H]2CCCCC2(F)F)C3)c1] | (S)-2-amino-6-(2,2-difluorocyclohexyl)- 4-(4-ethoxy-1H-pyrazol-1-yl)-6,7- dihydro-5H-pyrrolo[3,4-d]pyrimidin-5- one |

In some embodiments is a compound of Formula (I), or pharmaceutically acceptable salt thereof, wherein R¹ is ethoxy. In further embodiments, the compound of Formula (I), or pharmaceutically acceptable salt thereof, is selected from the group consisting of:

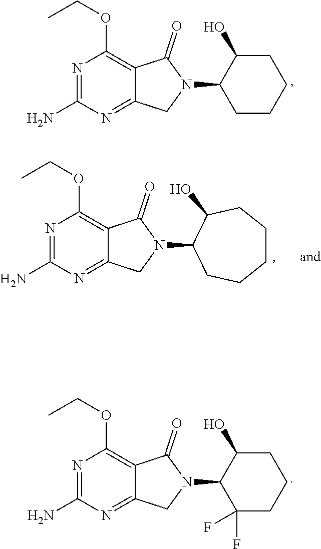

In some embodiments is a compound of Formula (I), or pharmaceutically acceptable salt thereof, wherein R¹ is methoxy. In further embodiments, the compound of Formula (I), or pharmaceutically acceptable salt thereof, is selected from the group consisting of:

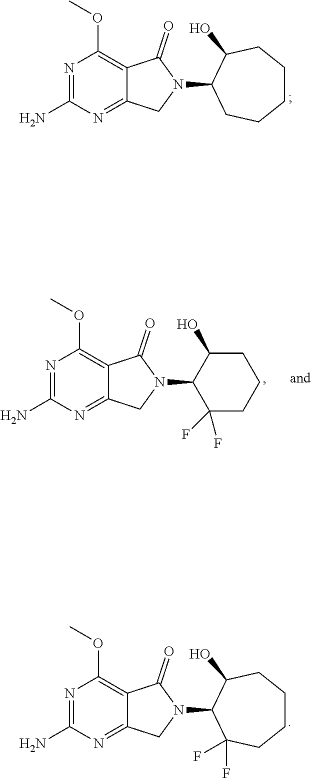 [SMILES: COc1nc(N)nc2c1C(=O)N([C@@H]1CCCCC[C@@H]1O)C2 ; COc1nc(N)nc2c1C(=O)N([C@H]1[C@@H](O)CCCC1(F)F)C2 ; COc1nc(N)nc2c1C(=O)N([C@H]1[C@@H](O)CCCCC1(F)F)C2]

In some embodiments is a compound of Formula (I), or pharmaceutically acceptable salt thereof, wherein R¹ is pyrazolyl or 4-ethoxy-1H-pyrazol-1-yl. In further embodiments, the compound of Formula (I), or pharmaceutically acceptable salt thereof, is selected from the group consisting of:

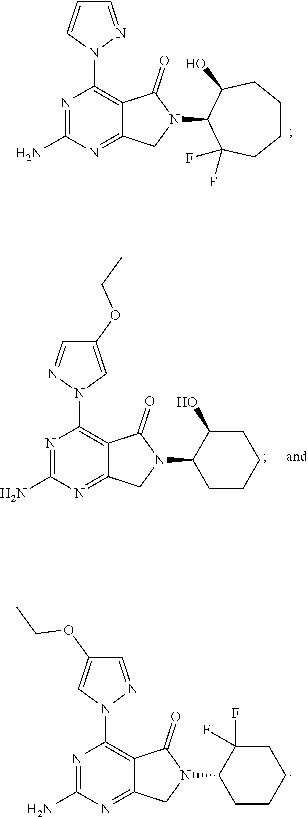 [SMILES: Nc1nc2c(c(-n3cccn3)n1)C(=O)N([C@H]1[C@@H](O)CCCCC1(F)F)C2 ; CCOc1cnn(-c2nc(N)nc3c2C(=O)N([C@@H]2CCCC[C@@H]2O)C3)c1 ; CCOc1cnn(-c2nc(N)nc3c2C(=O)N([C@H]2CCCCC2(F)F)C3)c1]

In some embodiments is a compound of Formula (I), or pharmaceutically acceptable salt thereof, wherein R¹ is —NHMe. In further embodiments, the compound of Formula (I), or pharmaceutically acceptable salt thereof, is

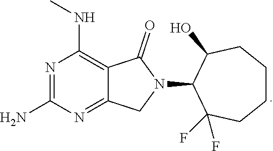 [SMILES: CNc1nc(N)nc2c1C(=O)N([C@H]1[C@@H](O)CCCCC1(F)F)C2]

In some embodiments, a compound is selected from the group consisting of: 2-amino-4-ethoxy-6-[(1R,2S)-2-hydroxycyclohexyl]-7H-pyrrolo[3,4-d]pyrimidin-5-one; 2-amino-4-ethoxy-6-((1R,2S)-2-hydroxycycloheptyl)-6,7-dihydro-5H-pyrrolo[3,4-d]pyrimidin-5-one; 2-amino-6-((1S,6S)-2,2-difluoro-6-hydroxycyclohexyl)-4-ethoxy-6,7-dihydro-5H-pyrrolo[3,4-d]pyrimidin-5-one; 2-amino-6-((1R,2S)-2-hydroxycycloheptyl)-4-methoxy-6,7-dihydro-5H-pyrrolo[3,4-d]pyrimidin-5-one; 2-amino-6-((1S,6S)-2,2-difluoro-6-hydroxycyclohexyl)-4-methoxy-6,7-dihydro-5H-pyrrolo[3,4-d]pyrimidin-5-one; 2-amino-6-((1S,7S)-2,2-difluoro-7-hydroxycycloheptyl)-4-methoxy-6,7-dihydro-5H-pyrrolo[3,4-d]pyrimidin-5-one; 2-amino-6-((1S,7S)-2,2-difluoro-7-hydroxycycloheptyl)-4-(methylamino)-6,7-dihydro-5H-pyrrolo[3,4-d]pyrimidin-5-one; 2-amino-6-((1S,7S)-2,2-difluoro-7-hydroxycycloheptyl)-4-(1H-pyrazol-1-yl)-6,7-dihydro-5H-pyrrolo[3,4-d]pyrimidin-5-one; 2-amino-6-((1S,2R)-2-hydroxycycloheptyl)-4-methoxy-6,7-dihydro-5H-pyrrolo[3,4-d]pyrimidin-5-one; 2-amino-4-ethoxy-6-((1S,2R)-2-hydroxycycloheptyl)-6,7-dihydro-5H-pyrrolo[3,4-d]pyrimidin-5-one; 2-amino-4-(4-ethoxy-1H-pyrazol-1-yl)-6-((1R,2S)-2-hydroxycyclohexyl)-6,7-dihydro-5H-pyrrolo[3,4-d]pyrimidin-5-one; and(S)-2-amino-6-(2,2-difluorocyclohexyl)-4-(4-ethoxy-1H-pyrazol-1-yl)-6,7-dihydro-5H-pyrrolo[3,4-d]pyrimidin-5-one.

In some embodiments, a compound is

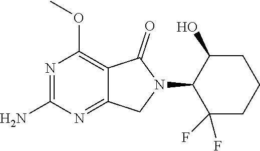 [SMILES: COc1nc(N)nc2c1C(=O)N([C@H]1[C@@H](O)CCCC1(F)F)C2]

or a pharmaceutically acceptable salt thereof.

In some embodiments, a compound or pharmaceutically acceptable salt thereof is selected from the group consisting of:

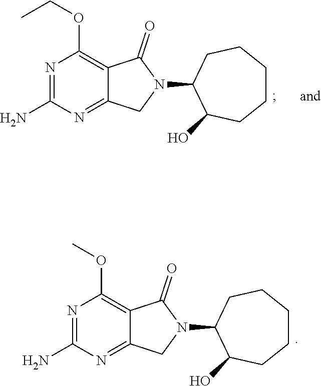 [SMILES: CCOc1nc(N)nc2c1C(=O)N([C@H]1CCCCC[C@H]1O)C2 ; COc1nc(N)nc2c1C(=O)N([C@H]1CCCCC[C@H]1O)C2]

It will be appreciated that a compound of Formula (I) may exist in different tautomeric forms. All possible tautomers, and pharmaceutically acceptable salts thereof, are contemplated to be within the scope of the present invention.

The compound of Formula (I) or pharmaceutically acceptable salt thereof may be in crystalline or amorphous forms. Furthermore, some of the crystalline forms may exist as polymorphs, all of which are included within the scope of the present invention. The most thermodynamically stable polymorphic form or forms of a compound of Formula (I) or a pharmaceutically acceptable salt thereof are of particular interest. In one aspect of the invention, a compound of Formula (I) or pharmaceutically acceptable salt thereof is crystalline.

### Compound Preparation

The compounds of Formula (I) or pharmaceutically acceptable salts thereof may be made by a variety of methods, including standard chemistry. The general procedures that can be used to synthesize the compounds of Formula (I) are described in reaction schemes below and are further illustrated in the Examples.

The compounds of Formula (Ia) may be prepared according to Scheme 1 by performing a reductive amination reaction of pyrimidine 5, dissolved in a suitable solvent, for example dichloromethane with an alkyl amine having the formula R⁴NH₂ and sodium triacetoxyborohydride. The reaction can be carried out at suitable temperature, for example, ambient temperature to 80° C., if required, to produce compounds of Formula 6. An oxidation reaction of the methylthiol of Formula 6 with oxone is carried out in a suitable solvent, for example acetonitrile:water, ratio 4:1, at ambient temperature to produce a mixture of the sulfone and sulfoxide of Formula 6. A displacement reaction with ammonia in a suitable solvent, for example 1,4-dioxane, at a suitable temperature, for example ambient to 80° C. can be used to produce compounds of Formula Ia.

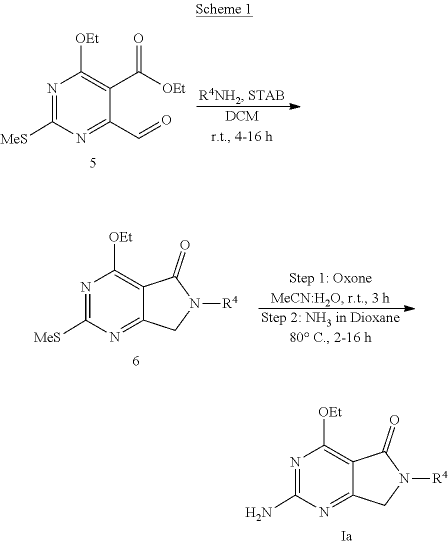

In some embodiments, R⁴ is

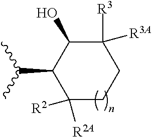

or

wherein R² is selected from hydroxyl, hydrogen or fluorine; R^{2A} is selected from hydrogen or fluorine; R³ and R^{3A} are each independently selected from hydrogen or methyl; and n is 1 or 2.

The compounds of Formula (Ib) may be prepared according to Scheme 2 by performing a reductive amination reaction of pyrimidine 12 dissolved in a suitable solvent, for example dichloromethane with an alkyl amine having the formula R⁴NH₂ and sodium triacetoxyborohydride. The reaction can be carried out at suitable temperature, for example, ambient temperature to 80° C. if required to produce compounds of Formula 13. Removal of the Boc protecting group using trifluoroacetic acid in dichloromethane at ambient temperature can be used to produce compounds of Formula Ib.

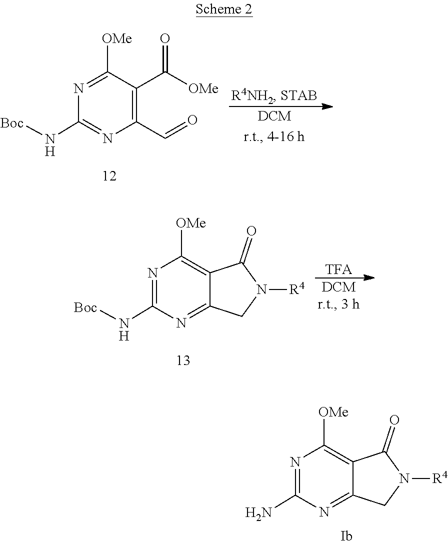

The compounds of Formula (Ic) may be prepared according to Scheme 3 by performing a reductive amination reaction of pyrimidine 16 dissolved in a suitable solvent, for example dichloromethane with an alkyl amine having the formula R⁴NH₂ and sodium triacetoxyborohydride. The reaction can be carried out at suitable temperature, for example, ambient temperature to 80° C. if required to produce compounds of Formula 17. Methylamine, in a suitable solvent, for example tetrahydrofuran was added to Formula 17 and heated at a suitable temperature, for example 80° C. to produce compounds of Formula 18. An oxidation reaction of the methylthiol of Formula 18 with oxone can be carried out in a suitable solvent, for example acetonitrile:water, ratio 4:1, at ambient temperature to produce a mixture of the sulfone and sulfoxide of Formula 18. A displacement reaction with ammonia in a suitable solvent, for example 1,4-dioxane, at a suitable temperature, for example ambient to 80° C. can be used to produce compounds of Formula Ic.

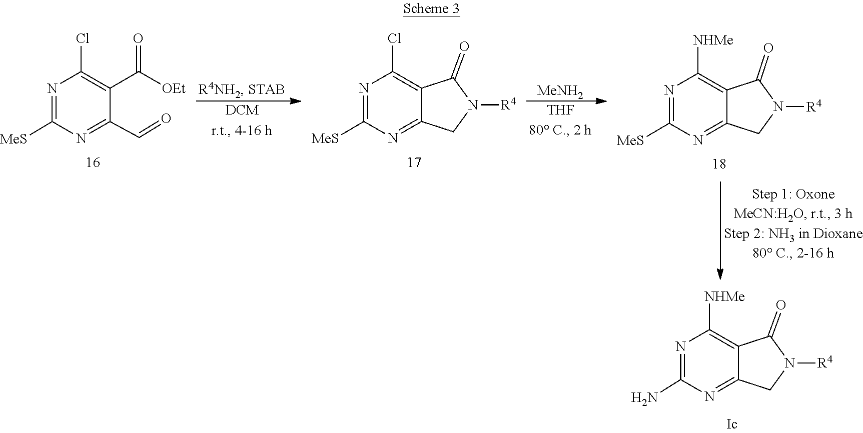

The compounds of Formula (Id) may be prepared according to Scheme 4 by performing a reductive amination reaction of pyrimidine 21 dissolved in a suitable solvent, for example dichloromethane with an alkyl amine having the formula R₁NH₂ and sodium triacetoxyborohydride. The reaction can be carried out at suitable temperature, for example, ambient temperature to 80° C., switching to a suitable solvent e.g. THF if required to produce compounds of Formula 22. An oxidation reaction of the methylthiol of Formula 22 with oxone can be carried out in a suitable solvent, for example acetonitrile:water, ratio 4:1, at ambient temperature to produce a mixture of the sulfone and sulfoxide of Formula 22. A displacement reaction with ammonia in a suitable solvent, for example 1,4-dioxane, at a suitable temperature, for example ambient to 80° C. can be used to produce compounds of Formula Id.

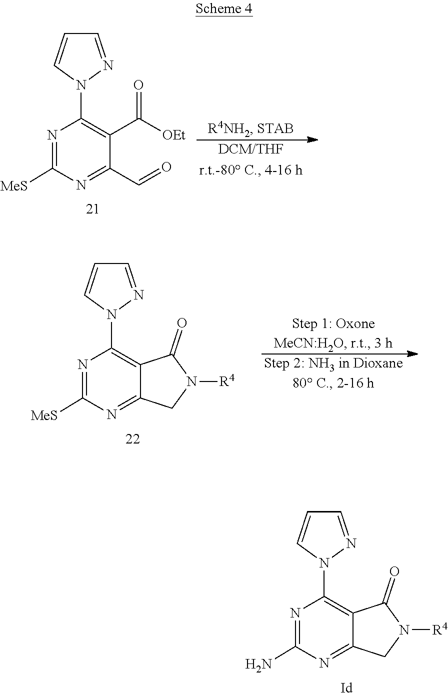

The compounds of Formula (Ie) may be prepared according to Scheme 5 by heating 4-ethoxy-1H-pyrazole. Hydrogen chloride in a suitable solvent, for example 1,4-dioxane and base, for example DIPEA at 105° C. to produce compounds of Formula 26. An oxidation reaction of the methylthiol of Formula 26 with oxone can be carried out in a suitable solvent, for example acetonitrile:water, ratio 4:1, at ambient temperature to produce a mixture of the sulfone and sulfoxide of Formula 26. A displacement reaction with ammonia in a suitable solvent, for example 1,4-dioxane, at a suitable temperature, for example ambient to 80° C. can be used to produce compounds of Formula Ie.

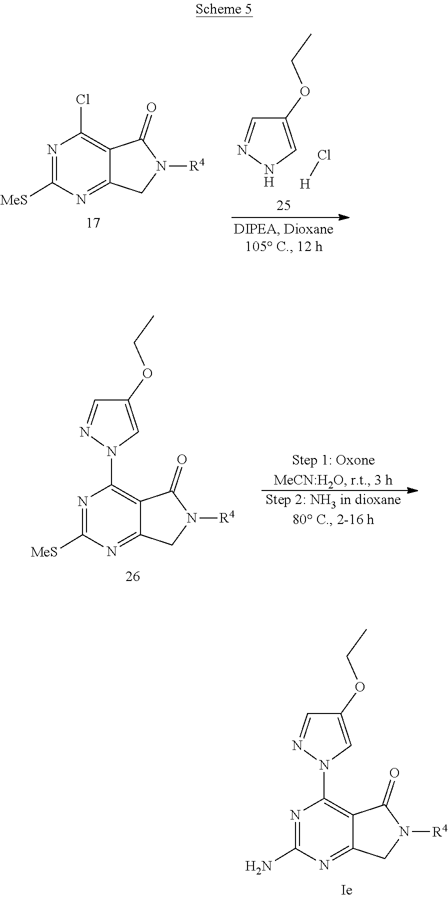

### Methods of Use and Treatment

In some embodiments, the invention relates to a compound of Formula (I), or a pharmaceutically acceptable salt thereof, for use in therapy.

In other embodiments, the invention relates to a compound of Formula (I), or a pharmaceutically acceptable salt thereof, for use in the treatment of a mycobacterial infection. A mycobacterial infection is one caused by infection with a mycobacterium.

In still other embodiments, the invention relates to a method of treatment of a mycobacterial infection in a human in need thereof, the treatment comprising administering to the human a therapeutically effective amount of a compound of Formula (I), or pharmaceutically acceptable salt thereof. As described herein, a mycobacterial infection is one caused by infection with a mycobacterium. The mycobacterium is as hereinbefore described.

In other embodiments, the invention relates to a method of treatment of a Mycobacterium tuberculosis infection.

In yet other embodiments, the invention relates to a method of treatment of a disease caused by infection with a mycobacterium in a mammal in need thereof, said treatment comprising administering to said mammal a therapeutically effective amount of a compound of Formula (I), or a pharmaceutically acceptable salt thereof.

In some embodiments, the disease is tuberculosis. Therefore, also described herein is a method of treatment of tuberculosis in a human in need thereof, said treatment comprising administering to said mammal a therapeutically effective amount of a compound of Formula (I), or a pharmaceutically acceptable salt thereof.

In still other embodiments, the invention relates to use of a compound of Formula (I), or a pharmaceutically acceptable salt thereof, in the manufacture of a medicament for use in the treatment of a mycobacterial infection or a disease caused by infection with a mycobacterium.

Also described herein is the use of a compound of Formula (I), or a pharmaceutically acceptable salt thereof, in the manufacture of a medicament for use in the treatment of tuberculosis.

Aminoacyl-tRNA synthetases are essential enzymes that transmit information from the genetic code to proteins in cells and are targets for antipathogen drug development. Aminoacyl-tRNA synthetases (aaRSs) attach specific amino acids to cognate tRNA molecules. During translation, each amino acid is carried by a specific tRNA to the translation site within the ribosome.

In some embodiments, a method of blocking or inhibiting aminoacyl-tRNA synthetases in a human in need thereof is provided. The method comprises administering to the human in need thereof a therapeutically effective amount of a compound of Formula (I), or a pharmaceutically acceptable salt thereof.

In some embodiments, a method of treating a mycobacterial infection in a human in need thereof is provided. The method comprising administering to the human in need thereof a therapeutically effective amount of a compound of Formula (I) or pharmaceutically acceptable salt thereof to block or inhibit aminoacyl-tRNA synthetases in the human. In one embodiment the aminoacyl tRNA synthetase is lysine aminoacyl tRNA synthetase.

The mycobacterium may be a member of one of the following groups of mycobacterium: Mycobacterium tuberculosis complex (MTC), Mycobacterium avium complex (MAC), Mycobacterium gordonae clade, Mycobacterium kansasii clade, Mycobacterium chelonae clade, Mycobacterium fortuitum clade, Mycobacterium parafortuitum clade or Mycobacterium vaccae clade. The mycobacterium may also be Mycobacterium ulcerans or Mycobacterium leprae.

In some embodiments, the mycobacterium is a member of the Mycobacterium tuberculosis complex (MTC).

Members of Mycobacterium tuberculosis complex (MTC) include Mycobacterium tuberculosis, Mycobacterium africanum, Mycobacterium bovis, Mycobacterium bovis BCG, Mycobacterium canetti, Mycobacterium caprae, Mycobacterium microti and Mycobacterium pinnipedii. These mycobacteria are causative agents of human and animal tuberculosis. Mycobacterium tuberculosis is the major cause of human tuberculosis.

In some embodiments, the infection is a Mycobacterium tuberculosis infection. In other words, the mycobacterial infection is caused by infection with Mycobacterium tuberculosis.

Members of Mycobacterium avium complex (MAC) include Mycobacterium avium, Mycobacterium avium paratuberculosis, Mycobacterium avium silaticum, Mycobacterium avium hominissuis, Mycobacterium columbiense and Mycobacterium indicus pranii.

Members of Mycobacterium gordonae clade include Mycobacterium asiaticum and Mycobacterium gordonae.

Members of Mycobacterium kansasii clade include Mycobacterium gastri and Mycobacterium kansasii.

Members of Mycobacterium chelonae clade include Mycobacterium abscessus, Mycobacterium bolletii and Mycobacterium chelonae.

Members of Mycobacterium fortuitum clade include Mycobacterium boenickei, Mycobacterium brisbanense, Mycobacterium cosmeticum, Mycobacterium fortuitum, Mycobacterium fortuitum subspecies acetamidolyticum, Mycobacterium houstonense, Mycobacterium mageritense, Mycobacterium neworleansense, Mycobacterium peregrinum, Mycobacterium porcinum, Mycobacterium senegalense and Mycobacterium septicum.

Members of Mycobacterium parafortuitum clade include Mycobacterium austroafricanum, Mycobacterium diernhoferi, Mycobacterium frederiksbergense, Mycobacterium hodleri, Mycobacterium neoaurum and Mycobacterium parafortuitum.

Therefore, the mycobacterial infection may be caused by infection with a mycobacterium selected from the following: Mycobacterium tuberculosis, Mycobacterium africanum, Mycobacterium bovis, Mycobacterium bovis BCG, Mycobacterium canetti, Mycobacterium caprae, Mycobacterium microti, Mycobacterium pinnipedii, Mycobacterium avium, Mycobacterium avium paratuberculosis, Mycobacterium avium silaticum, Mycobacterium avium hominissuis, Mycobacterium columbiense, Mycobacterium indicus pranii, Mycobacterium asiaticum, Mycobacterium gordonae, Mycobacterium gastri, Mycobacterium kansasii, Mycobacterium abscessus, Mycobacterium bolletii, Mycobacterium chelonae, Mycobacterium boenickei, Mycobacterium brisbanense, Mycobacterium cosmeticum, Mycobacterium fortuitum, Mycobacterium fortuitum subspecies acetamidolyticum, Mycobacterium houstonense, Mycobacterium mageritense, Mycobacterium neworleansense, Mycobacterium peregrinum, Mycobacterium porcinum, Mycobacterium senegalense, Mycobacterium septicum, Mycobacterium austroafricanum, Mycobacterium diernhoferi, Mycobacterium frederiksbergense, Mycobacterium hodleri, Mycobacterium neoaurum, Mycobacterium parafortuitum, Mycobacterium ulcerans and Mycobacterium leprae.

In other embodiments, the invention relates to a compound of Formula (I), or a pharmaceutically acceptable salt thereof, for use in the treatment of a disease caused by infection with a mycobacterium, where the mycobacterium is selected from those hereinbefore described. Diseases caused by infection with a mycobacterium include, but are not limited to, tuberculosis (e.g. from Mycobacterium tuberculosis), leprosy (e.g. from Mycobacterium leprae), Johne's disease (e.g. from Mycobacterium avium subspecies paratuberculosis), Buruli or Bairnsdale ulcer (e.g. from Mycobacterium ulceran), Crohn's disease (e.g. from Mycobacterium avium subspecies paratuberculosis), pulmonary disease or pulmonary infection, pneumonia, bursa, synovial, tendon sheaths, localized abscess, lymphadenitis, skin and soft tissue infections, Lady Windermere syndrome (e.g. from Mycobacterium avium complex (MAC)), MAC lung disease, disseminated Mycobacterium avium complex (DMAC), disseminated Mycobacterium avium intraceullulare complex (DMAIC), hot-tub lung (e.g. from Mycobacterium avium complex), MAC mastitis, MAC pyomyositis, or granuloma disease.

In some embodiments, the disease is tuberculosis. Thus, one aspect of the invention relates to a compound of Formula (I), or pharmaceutically acceptable salts thereof, for use in the treatment of tuberculosis.

### Formulations

The compound of Formula (I) or pharmaceutically acceptable salt thereof will normally, but not necessarily, be formulated into pharmaceutical formulations prior to administration to a patient. Accordingly, in another aspect there is provided a pharmaceutical formulation comprising a compound of Formula (I), or a pharmaceutically acceptable salt thereof, and a pharmaceutically acceptable excipient.

Pharmaceutical compositions may be administered by any appropriate route, for example by the oral (including buccal or sublingual), inhaled, intranasal, topical (including buccal, sublingual or transdermal), parenteral (including subcutaneous, intramuscular, intravenous or intradermal) route.

In particular, pharmaceutical compositions are administered via an oral route of administration. Suitable pharmaceutically acceptable excipients include the following types of excipients: carriers, diluents, fillers, binders, disintegrants, lubricants, glidants, granulating agents, coating agents, wetting agents, solvents, co-solvents, suspending agents, emulsifiers, sweeteners, flavouring agents, flavour-masking agents, colouring agents, anti-caking agents, humectants, chelating agents, plasticisers, viscosity increasing agents, antioxidants, preservatives, stabilisers, surfactants and buffering agents.

Suitable methods for formulating a compound of Formula (I) or a pharmaceutically acceptable salt thereof will be familiar to those skilled in the art, which are described in Remington: The Science and Practice of Pharmacy, 21^{st} Edition 2006.

Pharmaceutical compositions may be presented in unit dose forms containing a predetermined amount of active ingredient per unit dose. Preferred unit dosage compositions are those containing a daily dose or sub-dose, or an appropriate fraction thereof, of an active ingredient. Such unit doses may therefore be administered more than once a day. Preferred unit dosage compositions are those containing a daily dose or sub-dose (for administration more than once a day), as herein above recited, or an appropriate fraction thereof, of an active ingredient.

When a compound of Formula (I) or pharmaceutically acceptable salt thereof is used in the treatment of tuberculosis, they may be employed alone or in combination with a further therapeutic agent, such as a further anti-mycobacterial agent, in particular a further anti-tuberculosis agent and/or antiviral agent, including antiretroviral agents.

For example, the present invention relates to a combination of (a) a compound of Formula (I) or a pharmaceutically acceptable salt thereof, and (b) a further anti-tuberculosis agent. In an embodiment, the combination comprises two, three, four, five, six or seven additional anti-tuberculosis agents. For example, in the treatment of multidrug-resistant tuberculosis, it is common that combinations of four or more drugs are administered to patients. For example, in the treatment of drug-sensitive tuberculosis, it is common that combinations of three or four drugs are administered to patients.

The further anti-tuberculosis agent is an agent in development, approved or recommended for the treatment of tuberculosis and may be selected from isoniazid, rifampin (rifampicin), pyrazinamide, ethambutol, rifapentine, clofazimine, ethionamide, prothionamide, isoxyl, thiacetazone, rifabutin, 4-aminosalicylic acid (PAS), cycloserine, spectinamide 1810, a fluoroquinolone such as moxifloxacin, gatifloxacin or levofloxacin; a diarylquinoline such as bedaquiline (TMC207) or TBAJ-587 or TBAJ-876; nitroimidazo-oxazine PA-824, delamanid (OPC-67683), an oxazolidinone such as linezolid, tedizolid, radezolid, sutezolid (PNU-100480), posizolid (AZD-5847), Delpazolid (LCB01-0371) or TBI-223; SPR720, EMB analogue SQ109, OPC-167832, telacebec (Q203), spectinamide 1810, GSK3036656 (also known as GSK070), GSK2556286 (GSK-286), GSK3211830, GSK3778839, GSK3729098 (BVL-GSK098), GSK3653038, a benzothiazinone such as BTZ043 or macozinone (PBTZ169); an azaindole such as TBA-7371, a dihyrdocarbostyril derivative such as OPC-167832; a dinitrobenzamide, a beta-lactam such as meropenem, faropenem, ertapenem, tebipenem, sanfetrinem; a beta-lactam combination such as AUGMENTIN (amoxicillin-clavulanate), or an aminoglycoside such as kanamycin, amikacin, capreomycin or streptomycin.

In one embodiment the further anti-tuberculosis agent is an agent in development, approved or recommended for the treatment of tuberculosis and may be selected from isoniazid, rifampin, pyrazinamide, ethambutol, moxifloxacin, rifapentine, clofazimine, ethionamide, prothionamide, isoxyl, thiacetazone, a diarylquinoline such as bedaquiline (TMC207) or TBAJ-587, nitroimidazo-oxazine PA-824, delamanid (OPC-67683), an oxazolidinone such as linezolid, tedizolid, radezolid, sutezolid (PNU-100480), posizolid (AZD-5847) or TBI-223, EMB analogue SQ109, OPC-167832, GSK3036656 (also known as GSK070), GSK2556286, GSK3211830, a benzothiazinone such as BTZ043 or PBTZ169, an azaindole such as TBA-7371, a dinitrobenzamide, or a beta-lactam such as meropenem, faropenem, ertapenem, tebipenem or beta-lactam combinations such as AUGMENTIN (amoxicillin-clavulanate).

A combination according to the present invention may further comprise an antiviral agent, including an antitretroviral agents. In an aspect of the invention, the composition further comprises a therapeutically effective amount at least one other agent used for treatment of AIDS or HIV infection selected from nucleoside HIV reverse transcriptase inhibitors, non-nucleoside HIV reverse transcriptase inhibitors, HIV protease inhibitors, HIV fusion inhibitors, HIV attachment inhibitors, CCR5 inhibitors, CXCR4 inhibitors, HIV budding or maturation inhibitors, and HIV integrase inhibitors, and a pharmaceutically acceptable carrier.

Such antiretroviral agents may be selected from abacavir, atazanavir, bictegravir, cabotegravir, darunavir, delavirdine, didanosine, dideoxyinosine, dolutegravir, doravirine, efavirenz, elvitegravir, emtricitabine, etavirine, fosamprenavir, fostemsavir, indinavir, slatravir, lamivudine, lopinavir, maraviroc, nelfinavir, nevirapine, raltegravir, rilpiverine, ritonavir, saquinavir, stavudine, tipranavir, tenofovir, tenofovir alafenamide, tenofovir disoproxil fumarate, zalcitabine, and zidovudine.

The combinations may conveniently be presented for use in the form of a pharmaceutical composition or formulation. Therefore, also contemplated herein is a pharmaceutical composition comprising (a) a compound of Formula (I) or a pharmaceutically acceptable salt thereof, as herein described, together with (b) a further anti-tuberculosis agent and (c) optionally an antiviral agent including antiretroviral agents, and (d) one or more pharmaceutically acceptable excipients, as herein described.

A compound of Formula (I) or a pharmaceutically acceptable salt thereof and further therapeutic agent may be administered together in a unitary pharmaceutical composition including both compounds or separately in separate pharmaceutical compositions, each including one of the compounds in a sequential manner. Such sequential administration may be close in time (e.g. simultaneously) or remote in time. Furthermore, it does not matter if the compounds are administered in the same dosage form, e.g. one compound may be administered topically, and the other compound may be administered orally. The amount of a compound of Formula (I) or pharmaceutically acceptable salt thereof and the further therapeutically active agent(s) and the relative timings of administration will be selected in order to achieve the desired combined therapeutic effect.

When combined in the same composition it will be appreciated that the two compounds must be stable and compatible with each other and the other components of the composition and may be formulated for administration. When formulated separately they may be provided in any convenient composition, conveniently, in such a manner as known for such compounds in the art.

The combinations may be presented as a combination kit. By the term “combination kit” “or kit of parts” as used herein is meant the pharmaceutical composition or compositions that are used to administer the combination according to the invention. When both compounds are administered simultaneously, the combination kit can contain both compounds in a single pharmaceutical composition, such as a tablet, or in separate pharmaceutical compositions. When the compounds are not administered simultaneously, the combination kit will contain each compound in separate pharmaceutical compositions either in a single package in separate pharmaceutical compositions in separate packages.

The combination kit can also be provided by instruction, such as dosage and administration instructions. Such dosage and administration instructions can be of the kind that are provided to a doctor, for example by a drug product label, or they can be of the kind that are provided by a doctor, such as instructions to a patient.

During a treatment regime, it will be appreciated that administration of each compound may be repeated one or more times.

When the combination is administered separately in a sequential manner wherein one is administered first and the other second or vice versa, such sequential administration may be close in time or remote in time. For example, administration of the other agent several minutes to several dozen minutes after the administration of the first agent, and administration of the other agent several hours to several days after the administration of the first agent are included, wherein the lapse of time is not limited, For example, one agent may be administered once a day, and the other agent may be administered 2 or 3 times a day, or one agent may be administered once a week, and the other agent may be administered once a day and the like.

It will be clear to a person skilled in the art that, where appropriate, the other therapeutic ingredients(s) may be used in the form of salts, for example as alkali metal or amine salts or as acid addition salts, or prodrugs, or as esters, for example lower alkyl esters, or as solvates, for example hydrates, to optimise the activity and/or stability and/or physical characteristics, such as solubility, of the therapeutic ingredient. It will be clear also that, where appropriate, the therapeutic ingredients may be used in optically pure form.

When combined in the same composition it will be appreciated that the two compounds must be stable and compatible with each other and the other components of the composition and may be formulated for administration. When formulated separately they may be provided in any convenient composition, conveniently, in such a manner as known for such compounds in the art.

### EXAMPLES

The invention will now be illustrated by way of the following non-limiting examples. While particular embodiments of the invention are described below a skilled person will appreciate that various changes and modifications can be made. References to preparations carried out in a similar manner to, or by the general method of, other preparations, may encompass variations in routine parameters such as time, temperature, workup conditions, minor changes in reagents amounts, etc. In certain of the following Intermediates and Examples, starting materials are identified by reference to other Intermediate or Example numbers. This does not signify that the actual material (or “batch”) obtained from any particular Intermediate or Example was necessarily used in a subsequent step exemplified herein, but is used as a short-hand means of denoting the relevant compound name.

### Abbreviations

The following list provides definitions of certain abbreviations and symbols as used herein. It will be appreciated that the list is not exhaustive, but the meaning of those abbreviations and symbols not herein below defined will be readily apparent to those skilled in the art. In describing the invention, chemical elements are identified in accordance with the Periodic Table of the Elements.
ATP Adenosine Triphosphate
BAST Diethylaminosulfur trifluoride
CFU Colony Forming Unit
DCM Dichloromethane
DIPEA Diisopropylethylamine
DMSO-d₆ Deuterated Dimethylsulfoxide
ES+MS Positive electrospray ionization mass spectrometry
EtOAc Ethyl acetate
EtOH Ethanol
g grams
HPLC High Performance Liquid Chromatography
Hz Hertz
LDA Lithium diisopropylamide
M Molar concentration
MeOH Methanol
Min minutes
mL Millilitre
MtKRS Mycobacterium tuberculosis lysine tRNA synthestase
μL Microlitre
μM Micromolar
μm Micrometre
mmol Millimole
N Normal concentration
N₂ Nitrogen
nm namometre
NMR Nuclear Magnetic Resonance spectroscopy
Rₜ Retention time
r.t. room temperature
STAB Sodiumtriacetoxyborohydride
TEA Triethylamine
TFA Trifluoroacetic acid
VS versus

### Analytical Equipment

¹H NMR spectra were recorded on a Bruker Avance DPX 500 spectrometer, or a Bruker Avance DPX 400. Chemical shifts (δ) are expressed in ppm recorded using the residual solvent as the internal reference in all cases. Signal splitting patterns are described as singlet(s), doublet (d), triplet (t), quartet (q), multiplet (m), broad (br), or a combination thereof. Coupling constants (J) are quoted to the nearest 0.1 Hz.

All temperatures reported are in degrees centigrade.

Low resolution electrospray (ES) mass spectra were recorded on an Advion Compact Mass Spectrometer (CMS; model ExpressIon CMS) connected to Dionex Ultimate 3000 UPLC system with diode array detector.

HPLC chromatographic separations were conducted using a Waters XBridge C18 column, 2.1×50 mm, 3.5 μm particle size or Waters XSelect 2.1×30 mm, 2.5 μm particle size. The compounds were eluted with a gradient of 5 to 95% acetonitrile/water+0.1% Ammonia or +0.1% formic acid.

### Intermediates

The synthesis of the pyrimidine intermediate 5 provided in Scheme 1 may be prepared according to Intermediate Scheme 1.

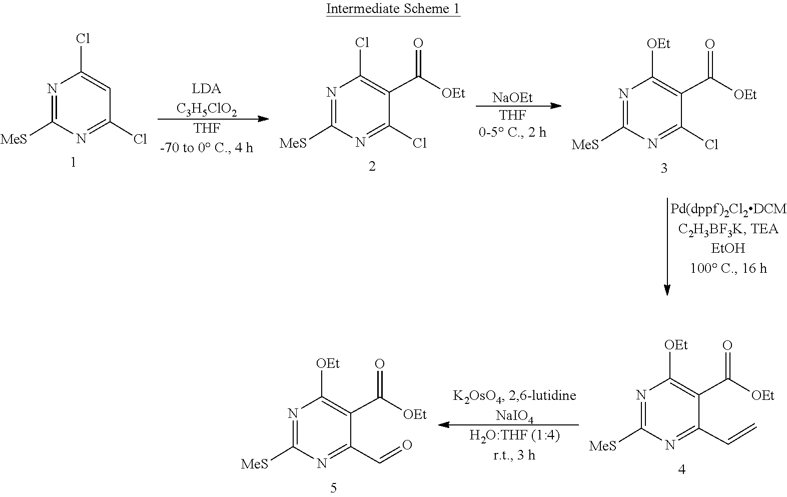 [SMILES: CSc1nc(Cl)cc(Cl)n1 ; CCOC(=O)c1c(Cl)nc(SC)nc1Cl ; CCOC(=O)c1c(Cl)nc(SC)nc1OCC ; CCOC(=O)c1c(C=O)nc(SC)nc1OCC ; C=Cc1nc(SC)nc(OCC)c1C(=O)OCC]

### Intermediate 2: Ethyl 4,6-dichloro-2-(methylthio)pyrimidine-5-carboxylate

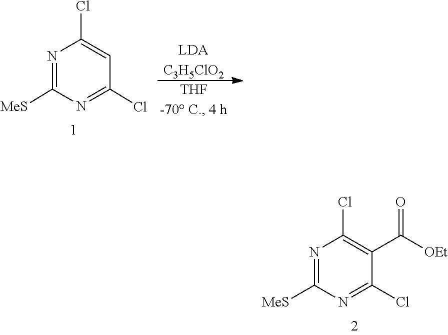 [SMILES: CSc1nc(Cl)cc(Cl)n1 ; CCOC(=O)c1c(Cl)nc(SC)nc1Cl]

To a solution of 4,6-dichloro-2-(methylthio)pyrimidine (50 g, 256 mmol, Intermediate 1, commercially available) in THF (350 mL) under nitrogen, cooled to −70° C., was added LDA (2M in THF/Hexanes, 192 mL, 384 mmol) dropwise, over a period of 50 min, keeping the temperature below −66° C. The resulting brown solution was stirred with cooling for 50 min. Ethyl chloroformate (37 mL, 384 mmol) was added dropwise over a period of 25 min and the mixture was stirred for 55 min, then allowed to warm to 0° C. for 70 min. The reaction was quenched by dropwise addition of brine and the aqueous layer was separated and extracted with EtOAc. The combined organics were washed with brine, dried over MgSO₄ and concentrated in vacuo. The crude material was dissolved in 97:3 heptane/EtOAc and passed through a pad of silica. The filtrate concentrated in vacuo and the residue dissolved in heptane (75 mL) and cooled in an ice bath. As soon as white crystals were observed, the flask was removed from the bath and crystallisation continued at r.t. The collected off-white solid was washed with cold heptane then dried under vacuum at r.t. to afford ethyl 4,6-dichloro-2-(methylthio)pyrimidine-5-carboxylate. The mother liquors were purified by column chromatography (silica gel; heptane-TMBE from 100/0 to 97/3) to afford ethyl 4,6-dichloro-2-(methylthio)pyrimidine-5-carboxylate (40.3 g, 59%) as a white solid. ¹H NMR (400 MHZ, CDCl₃) δ 4.48 (q, J=7.1 Hz, 2H), 2.61 (s, 3H), 1.43 (t, J=7.1 Hz, 3H). MS m/z [M+H]⁺=267.1.

### Intermediate 3: Ethyl 4-chloro-6-ethoxy-2-(methylthio)pyrimidine-5-carboxylate

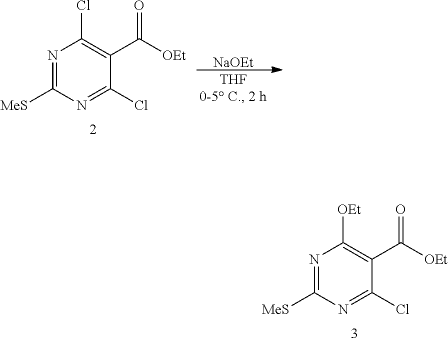 [SMILES: CCOC(=O)c1c(Cl)nc(SC)nc1Cl ; CCOC(=O)c1c(Cl)nc(SC)nc1OCC]

To a solution of ethyl 4,6-dichloro-2-methylsulfanyl-pyrimidine-5-carboxylate (34.8 g, 130 mmol, Intermediate 2) in THF (270 mL), under nitrogen, cooled to 2° C., was added sodium ethanolate (21% in EtOH, 49 mL, 130 mmol) dropwise over 55 min, and stirred for 45 min. The reaction was diluted with TBME and water. The combined organics were washed with brine, dried over Na₂SO₄ and concentrated in vacuo to afford ethyl 4-chloro-6-ethoxy-2-(methylthio)pyrimidine-5-carboxylate (37.4 g, quantitative) as an amber oil. The compound was used in subsequent steps without any purification. ¹H NMR (400 MHZ, CDCl₃) δ 4.51 (q, J=7.0 Hz, 2H), 4.42 (q, J=7.1 Hz, 2H), 2.57 (s, 3H), 1.42-1.37 (m, 6H). MS m/z [M+H]⁺=277.2.

### Intermediate 4: Ethyl 4-ethoxy-2-(methylthio)-6-vinylpyrimidine-5-carboxylate

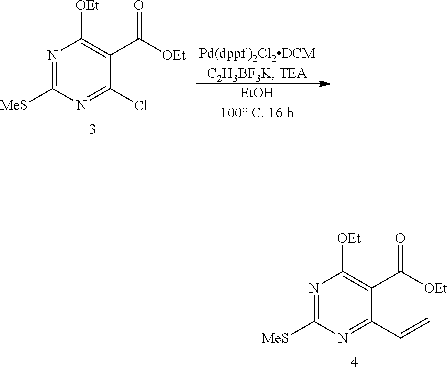 [SMILES: CCOC(=O)c1c(Cl)nc(SC)nc1OCC ; C=Cc1nc(SC)nc(OCC)c1C(=O)OCC]

To a solution of ethyl 4-chloro-6-ethoxy-2-(methylthio)pyrimidine-5-carboxylate (37.4 g, 135 mmol, Intermediate 3) and potassium vinyltrifluoroborate (23.4 g, 175 mmol) in EtOH (300 mL) was added TEA (21.4 mL, 154 mmol). Nitrogen was bubbled through the mixture for 10 min before addition of Pd(dppf)₂Cl₂·DCM (2.2 g, 2.70 mmol). The resulting mixture was heated at reflux under nitrogen for 3 h before the addition of potassium vinyltrifluoroborate (12 g, 89.6 mmol), TEA (11 mL, 78.9 mmol) and Pd(dppf)₂Cl₂·DCM (1.1 g, 1.35 mmol) and heating continued overnight. The reaction mixture was cooled and concentrated in vacuo. The residue was treated with 2N HCl, TBME was added and the combined organics were washed with sat. NaHCO₃ followed by brine, dried over MgSO₄ and concentrated in vacuo. The residue was dissolved in DCM and filtered through a pad of silica (silica gel; DCM), to afford ethyl 4-ethoxy-2-(methylthio)-6-vinylpyrimidine-5-carboxylate (18.5 g, 51%) as a pale brown oil. ¹H NMR (400 MHZ, CDCl₃) δ 6.80 (dd, J=16.8, 10.5 Hz, 1H), 6.64 (dd, J=16.8, 2.0 Hz, 1H), 5.64 (dd, J=10.5, 2.0 Hz, 1H), 4.46 (q, J=7.1 Hz, 2H), 4.38 (q, J=7.1 Hz, 2H), 2.56 (s, 3H), 1.39-1.35 (m, 6H). MS m/z [M+H]⁺=269.4.

### Intermediate 5: Ethyl 4-ethoxy-6-formyl-2-(methylthio)pyrimidine-5-carboxylate

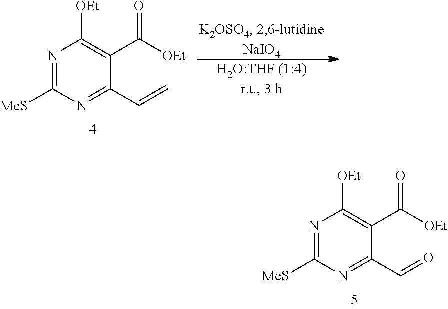 [SMILES: C=Cc1nc(SC)nc(OCC)c1C(=O)OCC ; CCOC(=O)c1c(C=O)nc(SC)nc1OCC]

To a solution of ethyl 4-ethoxy-2-methylsulfanyl-6-vinyl-pyrimidine-5-carboxylate (18.5 g, 68.9 mmol, Intermediate 4) in THF (400 mL) was added water (100 mL), sodium metaperiodate (41 g, 191.7 mmol) and potassium osmate (VI) oxide dihydrate (480 mg, 1.30 mmol) and stirred at r.t. for 3 h. The mixture was diluted with water and EtOAc and the combined organics were washed with brine, dried over MgSO₄ and filtered. The filtrate was treated with corn oil (5 mL) before the reaction was concentrated in vacuo and the residue filtered through a pad of silica (silica gel; heptane, followed heptane/EtOAc 90/10) to afford ethyl 4-ethoxy-6-formyl-2-(methylthio)pyrimidine-5-carboxylate (7.3 g, 39%) as a grey oil. ¹H NMR (400 MHZ, CDCl₃) δ 9.88 (s, 1H), 4.53 (q, J=7.1 Hz, 2H), 4.42 (q, J=7.1 Hz, 2H), 2.61 (s, 3H), 1.41 (t, J=7.1 Hz, 3H), 1.37 (t, J=7.1 Hz, 3H MS m/z [M+H]⁺=271.2.

The synthesis of the pyrimidine intermediate 12 provided in Scheme 2 may be prepared according to Intermediate Scheme 2.

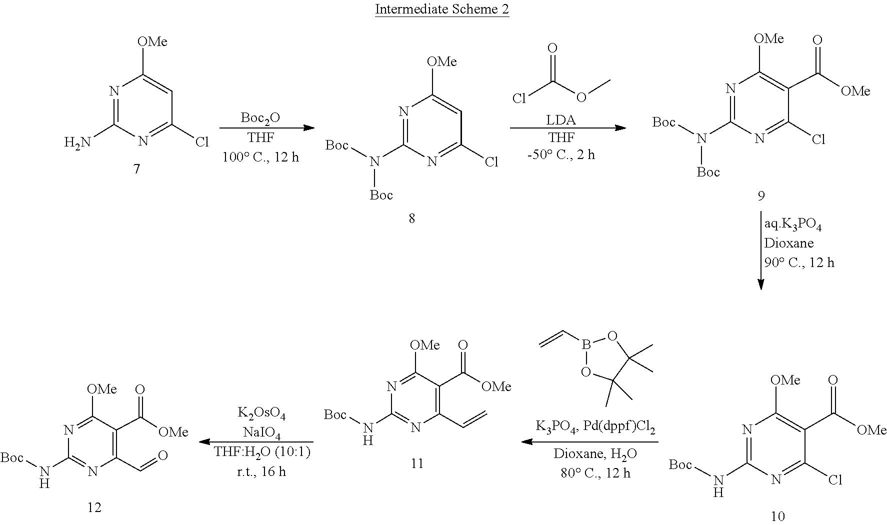

### Intermediate 8: Di-tert-butyl (4-chloro-6-methoxypyrimidin-2-yl) carbamate

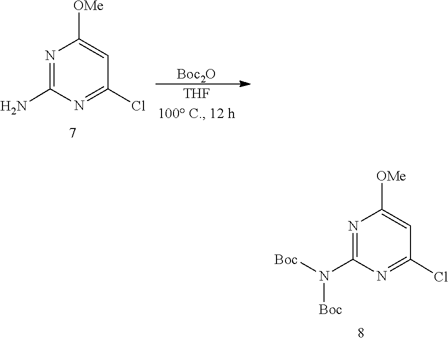 [SMILES: COc1cc(Cl)nc(N)n1 ; COc1cc(Cl)nc(N(C(=O)OC(C)(C)C)C(=O)OC(C)(C)C)n1]

To a mixture of 4-chloro-6-methoxypyrimidin-2-amine (50 g, 313 mmol, Intermediate 7, commercially available) and Boc₂O (273.5 g, 1.25 mol) in THF (500 mL) was added DMAP (5 g, 41 mmol) and the mixture was stirred at 100° C. for 12 h. The residue was poured into ice-water (w/w=1/1, 500 mL) and stirred for 5 min and extracted with EtOAc. The combined organics were washed with brine, dried over Na₂SO₄ and concentrated in vacuo. The residue was purified by column chromatography (silica gel; petroleum ether-EtOAc from 100/0 to 90/10) to afford di-tert-butyl (4-chloro-6-methoxypyrimidin-2-yl) carbamate (60 g, 53%) as a colourless oil. ¹H NMR (400 MHz, DMSO-d₆) δ 7.17 (s, 1H), 3.95 (s, 3H), 1.43 (s, 18H).

### Intermediate 9: Methyl 2-((di-tert-butoxycarbonyl)amino)-4-chloro-6-methoxypyrimidine-5-carboxylate

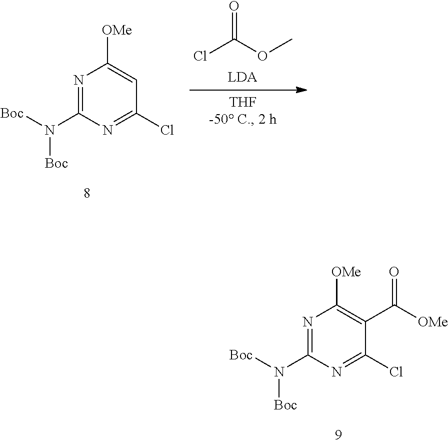

To a solution of di-tert-butyl (4-chloro-6-methoxypyrimidin-2-yl) carbamate (70 g, 194.5 mmol, Intermediate 8) in THF (1 L) was added LDA (2 M in THF/heptane, 194.5 mL, 389 mmol) dropwise at −60° C. under nitrogen. Then methyl chloroformate (28.7 mL, 370 mmol) was added into the mixture at −60° C. under nitrogen and the reaction was stirred at −40° C. for 2 h. The mixture was quenched with sat.NH₄Cl_{(aq)} and the aqueous phase was extracted with EtOAc. The combined organics were washed with brine, dried over Na₂SO₄, filtered and concentrated in vacuo. 4 batches of Intermediate 8 and 9 were combined and purified together by column chromatography (silica gel; Petroleum ether-EtOAc from 100/0 to 90/10) to afford methyl 2-((di-tert-butoxycarbonyl)amino)-4-chloro-6-methoxypyrimidine-5-carboxylate (290 g, 89%) as a yellow oil. ¹H NMR (400 MHZ, DMSO-d₆) δ 4.00 (s, 3H), 3.91 (s, 3H), 1.46 (s, 18H).

### Intermediate 10: Methyl 2-((tert-butoxycarbonyl)amino)-4-chloro-6-methoxypyrimidine-5-carboxylate

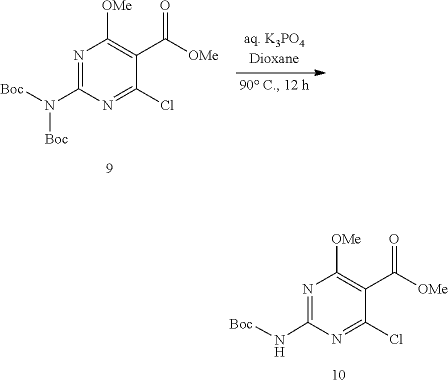 [SMILES: COC(=O)c1c(Cl)nc(N(C(=O)OC(C)(C)C)C(=O)OC(C)(C)C)nc1OC ; COC(=O)c1c(Cl)nc(NC(=O)OC(C)(C)C)nc1OC]

A mixture of methyl 2-((di-tert-butoxycarbonyl)amino)-4-chloro-6-methoxypyrimidine-5-carboxylate (250 g, 598 mmol, Intermediate 9) and K₃PO_{4(aq)} (2 M, 598 mL) in 1,4-dioxane (2 L) were stirred at 90° C. for 12 h. The residue was poured into ice-water (w/w=1/1, 500 mL) and stirred for 5 min. The aqueous phase was extracted with EtOAc and the combined organics were washed with brine, dried over Na₂SO₄, filtered and concentrated in vacuo. The residue was purified by column chromatography (silica gel; Petroleum ether-EtOAc from 100/0 to 90/10) to afford methyl 2-((tert-butoxycarbonyl)amino)-4-chloro-6-methoxypyrimidine-5-carboxylate (130 g, 68%) as a white solid. ¹H NMR (400 MHZ, DMSO-d₆) δ 10.57 (s, 1H), 4.02-3.96 (m, 3H), 3.84 (s, 3H), 1.47 (s, 9H).

### Intermediate 11: Methyl 2-((tert-butoxycarbonyl)amino)-4-methoxy-6-vinylpyrimidine-5-carboxylate

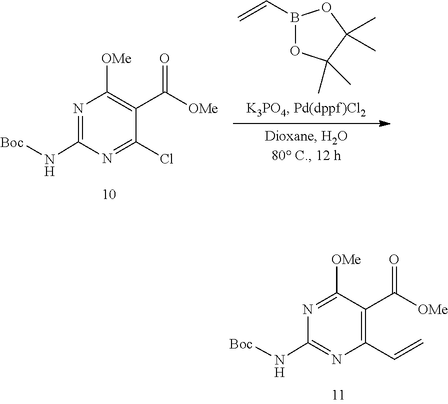 [SMILES: COC(=O)c1c(Cl)nc(NC(=O)OC(C)(C)C)nc1OC ; C=CB1OC(C)(C)C(C)(C)O1 ; C=Cc1nc(NC(=O)OC(C)(C)C)nc(OC)c1C(=O)OC]

To a solution of methyl 2-((tert-butoxycarbonyl)amino)-4-chloro-6-methoxypyrimidine-5-carboxylate (30 g, 69 mmol, Intermediate 10) in 1,4-dioxane (500 mL) was added 4,4,5,5-tetramethyl-2-vinyl-1,3,2-dioxaborolane (24 mL, 141.5 mmol), Pd(dppf)Cl₂ (3 g, 4.10 mmol) and K₃PO₄ (30 g, 141 mmol) as a solution in H₂O (100 mL) at 25° C. under nitrogen and the reaction was stirred at 80° C. for 12 h. The reaction mixture was poured into ice-water (w/w=1/1, 50 mL) and stirred for 5 min. The aqueous phase was extracted with EtOAc and the combined organics were washed with brine, dried over Na₂SO₄, filtered and concentrated in vacuo. The residue was purified by column chromatography (silica gel; Petroleum ether-EtOAc from 100/0 to 90/10) to afford methyl 2-((tert-butoxycarbonyl)amino)-4-methoxy-6-vinylpyrimidine-5-carboxylate (14.2 g, 57%) as a yellow solid. ¹H NMR (400 MHZ, DMSO-d₆) δ 10.11 (s, 1H), 6.83-6.68 (m, 1H), 6.64-6.52 (m, 1H), 5.78-5.67 (m, 1H), 3.94 (s, 3H), 3.84 (s, 3H), 1.48 (s, 9H).

### Intermediate 12: Methyl 2-((tert-butoxycarbonyl)amino)-4-formyl-6-methoxypyrimidine-5-carboxylate

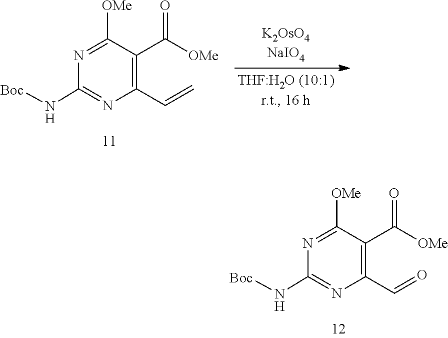 [SMILES: C=Cc1nc(NC(=O)OC(C)(C)C)nc(OC)c1C(=O)OC ; COC(=O)c1c(C=O)nc(NC(=O)OC(C)(C)C)nc1OC]

To a solution of methyl 2-((tert-butoxycarbonyl)amino)-4-formyl-6-methoxypyrimidine-5-carboxylate (10 g, 32.33 mmol, Intermediate 11) in THF (244.5 mL) and Water (24.5 mL) was added 2,6-lutidine (7.5 mL, 64.66 mmol), dipotassium dioxido (dioxo) osmium dihydrate (417 mg, 1.13 mmol) and sodium (meta) periodate (27.7 g, 129.32 mmol) and left overnight at r.t. Corn oil (10 mL) was added and stirred for 60 min. The reaction was concentrated in vacuo and the residue partitioned between EtOAc and water. The combined organics were washed with brine, dried over MgSO₄ and concentrated in vacuo. The residue was purified by column chromatography (silica gel; heptane-EtOAc from 100/0 to 50/50) to afford methyl 2-(tert-butoxycarbonylamino)-4-formyl-6-methoxy-pyrimidine-5-carboxylate (3.6 g, 36%) as a green viscous oil. ¹H NMR (500 MHZ, CDCl₃) δ 9.9 (s, 1H), 4.06 (s, 3H), 3.93 (s, 3H), 1.56 (s, 9H). m/z (ESI): 212.2 [M-BOC]⁺

The synthesis of the pyrimidine intermediate 16 provided in Scheme 3 may be prepared according to Intermediate Scheme 3.

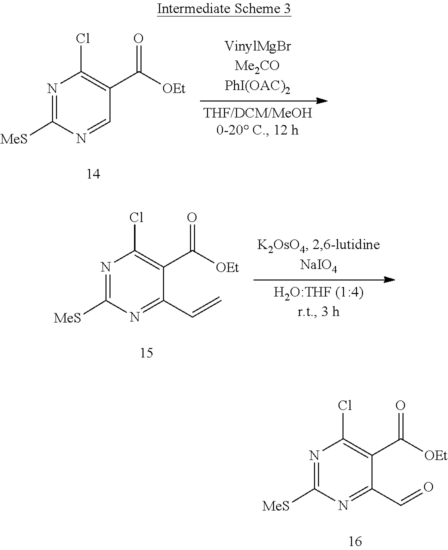 [SMILES: CCOC(=O)c1cnc(SC)nc1Cl ; C=Cc1nc(SC)nc(Cl)c1C(=O)OCC ; CCOC(=O)c1c(Cl)nc(SC)nc1C=O]

### Intermediate 15: Ethyl 4-chloro-2-(methylthio)-6-vinylpyrimidine-5-carboxylate

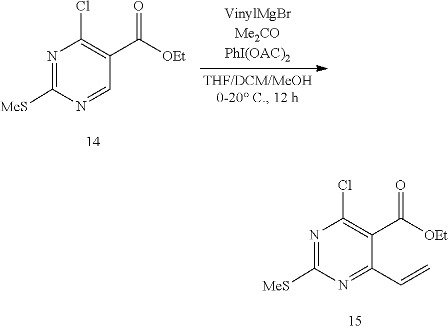 [SMILES: CCOC(=O)c1cnc(SC)nc1Cl ; C=Cc1nc(SC)nc(Cl)c1C(=O)OCC]

To a solution of ethyl 4-chloro-2-(methylthio)pyrimidine-5-carboxylate (150 g, 644.64 mmol, Intermediate 14) in THF (1 L), cooled to 0° C. was added vinylmagnesiumbromide (1 M, 967 mL) dropwise before the reaction mixture was stirred at 20° C. for 30 min. Acetone (38 mL, 515.71 mmol) was then added dropwise at 0° C. The reaction was concentrated in vacuo and the residue was dissolved in DCM (1.5 L) and MeOH (450 mL). PhI(OAc)₂ (311.5 g, 966.96 mmol) was added and the reaction mixture stirred at 20° C. for 12 h. The reaction mixture was poured into NaHCO_{3(aq)} (2 L) and stirred for 1 h. Then the mixture was extracted with DCM and the combined organic layers were washed with brine, dried over Na₂SO₄ and concentrated in vacuo. The residue was purified by column chromatography (silica gel; petroelum ether-EtOAc from 100/0 to 98/2) to afford ethyl 4-chloro-2-(methylthio)-6-vinylpyrimidine-5-carboxylate (135 g, 40%) as a brown oil. ¹H NMR (400 MHZ, CDCl₃) δ 6.79-6.67 (m, 2H), 5.77 (dd, J=9.3, 2.9 Hz, 1H), 4.45 (q, J=7.1 Hz, 2H), 2.60 (s, 3H), 1.41 (t, J=7.2 Hz, 3H). MS m/z 259.0 [M+H]⁺.

### Intermediate 16: Ethyl 4-chloro-6-formyl-2-(methylthio)pyrimidine-5-carboxylate

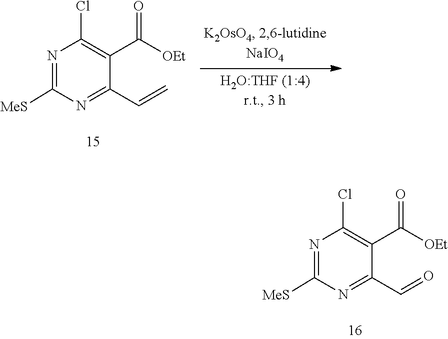 [SMILES: C=Cc1nc(SC)nc(Cl)c1C(=O)OCC ; CCOC(=O)c1c(Cl)nc(SC)nc1C=O]

To a solution of ethyl 4-chloro-2-(methylthio)-6-vinylpyrimidine-5-carboxylate (20 g, 77.30 mmol, Intermediate 15) in 1,4-dioxane (500 mL) and H₂O (75 mL) was added a solution of OsO₄ (4 wt. % in water, 401 μL, 7.73 mmol) and 2,6-lutidine (22.5 mL, 193.26 mmol) in H₂O (75 mL) dropwise at 0° C., the mixture was then stirred at 10° C. for 5 min, before NaIO₄ (83 g, 386.51 mmol) was added into the mixture in batches. The reaction was stirred at 25° C. for 2 h. The reaction mixture was poured into ice-water (w/w=1/1) (2000 mL) and stirred for 30 min. The aqueous phase was extracted with EtOAc and the combined organics were washed with brine, dried over Na₂SO₄ and concentrated in vacuo. The residue was purified by column chromatography (silica gel; petroelum ether-EtOAc from 100/0 to 95/5) to afford ethyl 4-chloro-6-formyl-2-(methylthio)pyrimidine-5-carboxylate. The reaction was repeated in parallel on the same scale three additional times to afford ethyl 4-chloro-6-formyl-2-(methylthio)pyrimidine-5-carboxylate 16 (32.45 g, 39%) as a colourless oil. ¹H NMR (400 MHZ, CDCl₃) δ 9.89 (s, 1H), 4.48 (q, J=7.2 Hz, 2H), 2.65 (s, 3H), 1.40 (t, J=7.2 Hz, 3H). MS m/z [M+H]⁺ 261.0.

The synthesis of the pyrimidine intermediate 21 provided in Scheme 4 may be prepared according to Intermediate Scheme 4.

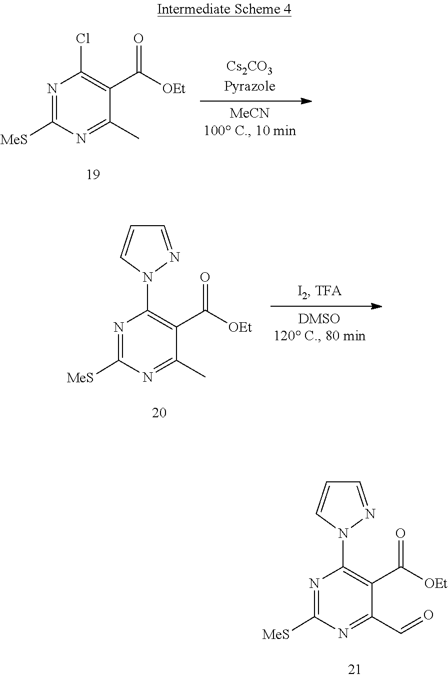 [SMILES: CCOC(=O)c1c(C)nc(SC)nc1Cl ; CCOC(=O)c1c(C)nc(SC)nc1-n1cccn1 ; CCOC(=O)c1c(C=O)nc(SC)nc1-n1cccn1]

### Intermediate 20: Ethyl 2-methyl-4-(methylthio)-6-(1H-pyrazol-1-yl)benzoate

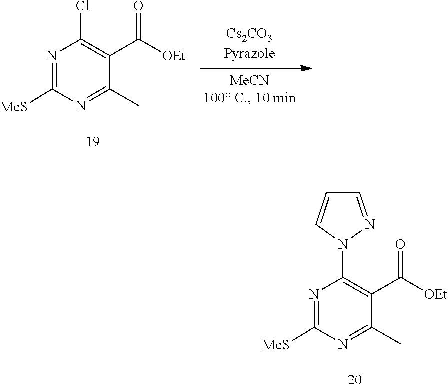 [SMILES: CCOC(=O)c1c(C)nc(SC)nc1Cl ; CCOC(=O)c1c(C)nc(SC)nc1-n1cccn1]

Cs₂CO₃ (3.36 g, 10.35 mmol) and pyrazole (705 mg, 10.35 mmol) were added to a solution of ethyl 2-chloro-6-methyl-4-(methylthio)benzoate (2.45 g, 10.35 mmol, Intermediate 19) in MeCN (39 mL) and the reaction subjected to microwave conditions for 10 min at 100° C. The reaction was diluted with water and DCM, passed through a hydrophobic frit and concentrated in vacuo. The residue was purified by column chromatography (0-40% EtOAc in heptane) and combined to afford ethyl 2-methyl-4-(methylthio)-6-(1H-pyrazol-1-yl)benzoate in (886 mg, 31%) as a yellow solid. ¹H NMR (500 MHz, DMSO-d₆) δ 8.68 (d, J=2.8 Hz, 1H), 7.88 (d, J=1.5 Hz, 1H), 6.65 (dd, J=2.8, 1.6 Hz, 1H), 4.30 (q, J=7.1 Hz, 2H), 2.61 (s, 3H), 2.45 (s, 3H), 1.24 (t, J=7.1 Hz, 3H). MS m/z [M+H]⁺=279.0.

### Intermediate 21: Ethyl 2-formyl-4-(methylthio)-6-(1H-pyrazol-1-yl)benzoate

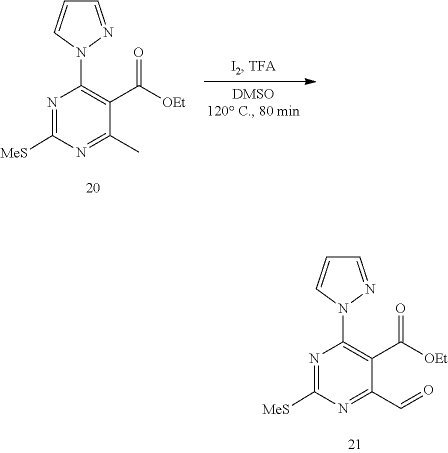 [SMILES: CCOC(=O)c1c(C)nc(SC)nc1-n1cccn1 ; CCOC(=O)c1c(C=O)nc(SC)nc1-n1cccn1]

I₂ (403 mg, 1.56 mmol) and TFA (0.48 mL, 6.34 mmol) were added to a solution of ethyl 2-methyl-4-(methylthio)-6-(1H-pyrazol-1-yl)benzoate (884 mg, 3.17 mmol, Intermediate 20) in anhydrous DMSO (60 mL) and heated to 120° C. for 80 min, stopped, cooled to r.t. and partitioned between EtOAc and sat. NaHCO₃, dried over Na₂SO₄ and concentrated in vacuo and purified by column chromatography (0-50% EtOAc in heptane) to afford ethyl 2-formyl-4-(methylthio)-6-(1H-pyrazol-1-yl)benzoate (365 mg, 39%) as a white solid. ¹H NMR (500 MHZ, CDCl₃) δ 9.93 (s, 1H), 8.57 (d, J=2.8 Hz, 1H), 7.75 (d, J=1.5 Hz, 1H), 6.50 (dd, J=2.8, 1.6 Hz, 1H), 4.51 (q, J=7.1 Hz, 2H), 2.68 (s, 3H), 1.40 (t, J=7.1 Hz). MS m/z [M+H]⁺=293.1.

The synthesis of the pyrazole intermediate 25 provided in Scheme 5 may be prepared according to Intermediate Scheme 5.

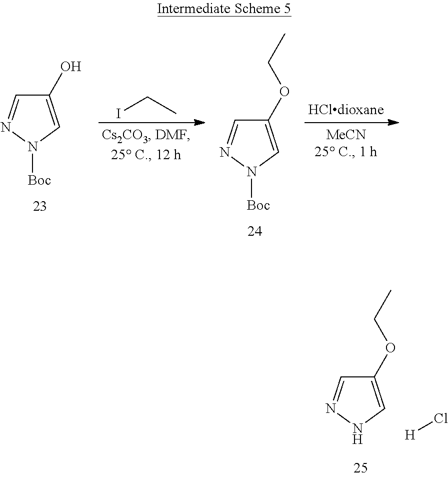

### Intermediate 24: Tert-butyl 4-ethoxy-1H-pyrazole-1-carboxylate

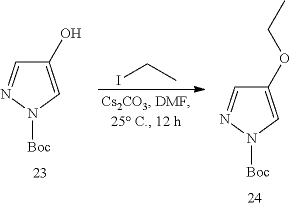

To a mixture of iodoethane (2.17 mL, 27.2 mmol) and tert-butyl 4-hydroxypyrazole-1-carboxylate (1 g, 5.43 mmol, Intermediate 23, commercially available) in DMF (20 mL) was added Cs₂CO₃ (5.31 g, 16.3 mmol) in one portion at 25° C. under N₂. The mixture was stirred at 25° C. for 12 h. The reaction was diluted with water and EtOAc and the combined organics were washed with brine, dried over Na₂SO₄, filtered and concentrated in vacuo. The residue was purified by column chromatography (silica gel; petroleum ether-EtOAc 100/0 to 67/33) to afford tert-butyl 4-ethoxy-1H-pyrazole-1-carboxylate (1.1 g, 95%) as a yellow solid. ¹H NMR (400 MHz, DMSO-d₆) δ 7.86 (s, 1H), 7.64 (s, 1H), 3.96 (q, J=7.2 Hz, 2H), 1.56 (s, 9H), 1.28 (t, J=7.2 Hz, 3H).

### Intermediate 25: 4-Ethoxy-1H-pyrazole·Hydrogen chloride

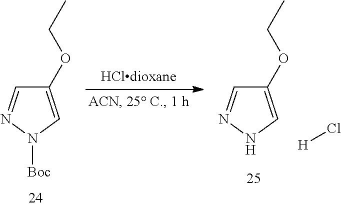

To a solution of tert-butyl 4-ethoxy-1H-pyrazole-1-carboxylate (1.0 g, 4.71 mmol, Intermediate 24) in ACN (5.0 mL) was added HCl·EtOAc (4 M, 5.0 mL) in one portion at 25° C. under N₂. The mixture was stirred at 25° C. for 1 h. The mixture was filtered and concentrated in vacuo to afford 4-ethoxy-1H-pyrazole·Hydrogen chloride (700 mg, quantitative) as a white solid. ¹H NMR (400 MHZ, DMSO-d₆) δ 12.51 (br s, 2H), 7.70 (s, 2H), 3.93 (q, J=6.8 Hz, 3H), 1.27 (t, J=6.8 Hz, 2H).

### General Procedure A: Cyclisation of Intermediates (6a-c, 13a-b, 17a-c, and 22a)

Referring to the synthetic approaches outlined in Schemes 1-4, the cyclisation reactions may be performed using General Procedure A. General Procedure A may be performed as follows: To a solution of the relevant aldehyde (1 eq) in DCM (0.13-0.78 M) with a spatula of MgSO₄ was added the amine (R₁NH₂, 1-1.2 equiv). If the amine is salted, then DIPEA (1 eq) is also added. The reaction is stirred for 5 min-1 h at r.t. before the addition of STAB (3 eq) and left overnight at r.t. to afford the uncyclized intermediate. The reaction was diluted water and passed through a hydrophobic frit and the residue taken up in THF (0.06-0.9 M) heated at 50-110° C. for 3 h-overnight. The reaction was diluted with water and DCM, passed through a hydrophobic frit and concentrated in vacuo before purification by column chromatography (silica gel; heptane-EtOAc from 100/0 to 0/100).

### Intermediate 6a: 4-Ethoxy-6-((1R,2S)-2-hydroxycyclohexyl)-2-(methylthio)-6,7-dihydro-5H-pyrrolo[3,4-d]pyrimidin-5-one

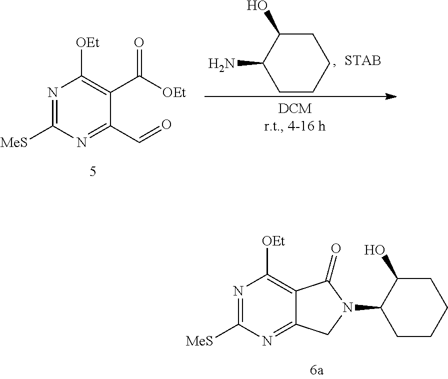 [SMILES: CCOC(=O)c1c(C=O)nc(SC)nc1OCC ; N[C@@H]1CCCC[C@@H]1O ; CCOc1nc(SC)nc2c1C(=O)N([C@@H]1CCCC[C@@H]1O)C2]

Following general procedure A, compound 6a was obtained from ethyl 4-ethoxy-6-formyl-2-(methylthio)pyrimidine-5-carboxylate (1.2 g, 4.44 mmol, Intermediate 5), (1S,2R)-2-aminocyclohexanol hydrochloride (673 mg, 4.44 mmol) and DIPEA (0.77 mL, 4.44 mmol) in DCM (20 mL) for 1 h, followed by the addition of STAB (2.82 g, 13.31 mmol). The uncyclized intermediate was then heated at 40° C. for 8 h in DCM (20 mL). 4-ethoxy-6-((1R,2S)-2-hydroxycyclohexyl)-2-(methylthio)-6,7-dihydro-5H-pyrrolo[3,4-d]pyrimidin-5-one (1.53 g. quantitative) as a pale-yellow oil. ¹H NMR (400 MHz, DMSO-d₆) δ 4.73 (d, J=4.1 Hz, 1H), 4.61 (d, J=19.6 Hz, 1H), 4.57-4.50 (m, 2H), 4.41 (d, J=19.6 Hz, 1H), 3.95-3.93 (m, 2H), 2.56 (s, 3H), 1.99-1.89 (m, 1H), 1.77-1.67 (m, 2H), 1.64-1.47 (m, 3H), 1.39-1.29 (m, 5H). MS m/z [M+H]⁺=324.4.

### Intermediate 6b: 6-(2-Hydroxycycloheptyl)-4-ethoxy-2-(methylthio)-6,7-dihydro-5H-pyrrolo[3,4-d]pyrimidin-5-one

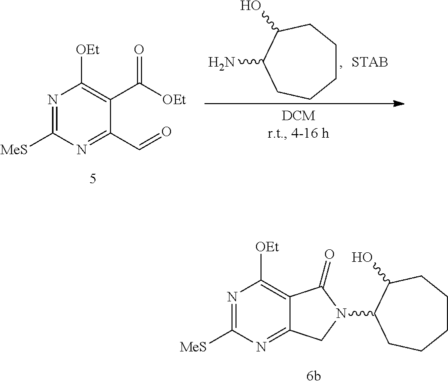 [SMILES: CCOC(=O)c1c(C=O)nc(SC)nc1OCC ; NC1CCCCCC1O ; CCOc1nc(SC)nc2c1C(=O)N(C1CCCCCC1O)C2]

Following general procedure A, compound 6b was obtained from ethyl 4-formyl-6-ethoxy-2-(methylthio)pyrimidine-5-carboxylate (1.23 g, 4.55 mmol, Intermediate 5) and 2-aminocycloheptan-1-ol (650 mg, 5 mmol, Intermediate 27) in DCM (50 mL) for 5 min, followed by STAB (2.9 g, 13.65 mmol). Cyclisation was achieved in THF (50 mL) at 50° C., overnight to afford the desired intermediate 6-(2-hydroxycycloheptyl)-4-ethoxy-2-(methylthio)-6,7-dihydro-5H-pyrrolo[3,4-d]pyrimidin-5-one (608 mg, 40%) as a yellow solid. ¹H NMR (400 MHZ, DMSO-d₆) δ 4.77 (d, J=4.7 Hz, 1H), 4.61-4.51 (m, 3H), 4.34 (d, J=19.7 Hz, 1H), 4.10-4.06 (m, 1H), 3.99-3.95 (m, 1H), 2.56 (s, 3H), 2.06-1.97 (m, 1H), 1.75-1.63 (m, 3H), 1.61-1.51 (m, 4H), 1.50-1.39 (m, 2H) 1.35 (t, J=7.1 Hz, 3H). MS m/z [M+H]⁺=293.16.

### Intermediate 6c: 6-((1S,6S)-2,2-difluoro-6-hydroxycyclohexyl)-4-ethoxy-2-(methylthio)-6,7-dihydro-5H-pyrrolo[3,4-d]pyrimidin-5-one

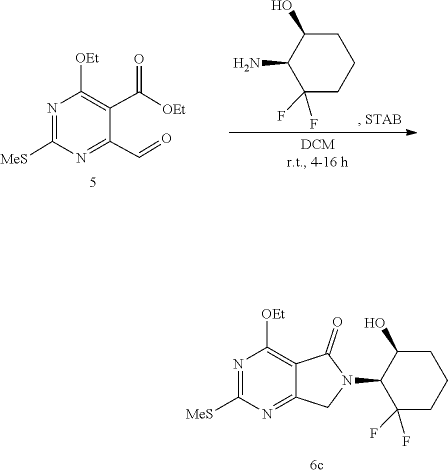 [SMILES: CCOC(=O)c1c(C=O)nc(SC)nc1OCC ; N[C@H]1[C@@H](O)CCCC1(F)F ; CCOc1nc(SC)nc2c1C(=O)N([C@H]1[C@@H](O)CCCC1(F)F)C2]

Following general procedure A, compound 6c was obtained from ethyl 4-formyl-6-ethoxy-2-(methylthio)pyrimidine-5-carboxylate (87 mg, 0.32 mmol, Intermediate 5), (1S,2S)-2-amino-3,3-difluoro-cyclohexanol (53 mg, 0.35 mmol) and STAB (204 mg, 0.96 mmol) in DCM (5 mL), overnight. Cyclisation was achieved in THF (5 mL), refluxed for 3 h to afford the desired intermediate 6-((1S,6S)-2,2-difluoro-6-hydroxycyclohexyl)-4-ethoxy-2-(methylthio)-6,7-dihydro-5H-pyrrolo[3,4-d]pyrimidin-5-one (75 mg, 65%) as a white solid. ¹H NMR (400 MHZ, DMSO-d₆) δ 4.77 (d, J=19.6 Hz, 1H), 4.46 (dd, J=19.6, 2.1 Hz, 1H), 4.41 (q, J=7.1 Hz, 2H), 4.36 (dt, J=26.0, 3.4 Hz, 1H), 4.15 (br s, 1H), 2.12-2.01 (m, 1H), 1.85-1.50 (m, 5H), 1.27 (t, J=7.1 Hz, 3H). MS m/z [M+H]⁺=360.5.

### Intermediate 13a: Tert-butyl (6-((1S,6S)-2,2-difluoro-6-hydroxycyclohexyl)-4-methoxy-5-oxo-6,7-dihydro-5H-pyrrolo[3,4-d]pyrimidin-2-yl) carbamate

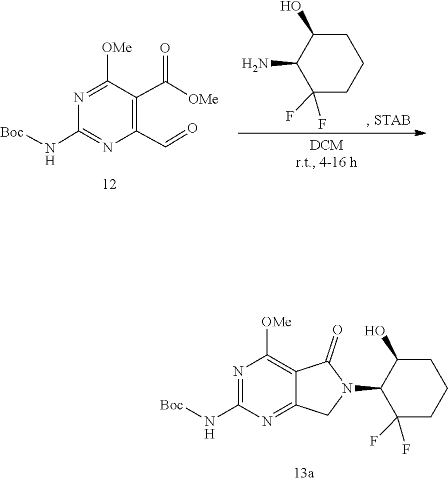 [SMILES: COC(=O)c1c(C=O)nc(NC(=O)OC(C)(C)C)nc1OC ; N[C@H]1[C@@H](O)CCCC1(F)F ; COc1nc(NC(=O)OC(C)(C)C)nc2c1C(=O)N([C@H]1[C@@H](O)CCCC1(F)F)C2]

Following general procedure A, compound 13a was obtained from methyl 2-((tert-butoxycarbonyl)amino-4-formyl-6-methoxypyrimidine-5-carboxylate (250 mg, 0.80 mmol, Intermediate 12), (1S,2S)-2-amino-3,3-difluorocyclohexan-1-ol acetate (186 mg, 0.88 mmol), DCM (8 mL) and DIPEA (0.13 mL, 0.96 mmol) for 5 min, followed by STAB (510 mg, 2.41 mmol). Cyclisation was achieved in THF (5 mL) at reflux for 7 h to afford the desired intermediate tert-butyl (6-((1S,6S)-2,2-difluoro-6-hydroxycyclohexyl)-4-methoxy-5-oxo-6,7-dihydro-5H-pyrrolo[3,4-d]pyrimidin-2-yl)carbamatein (180 mg, 54%) as a white solid. ¹H NMR (400 MHZ, CDCl₃) δ 7.64 (s, 2H), 5.17 (d, J=20.3 Hz, 1H), 4.82 (d, J=20.3 Hz, 1H), 4.61-4.53 (m, 1H), 4.37 (br s, 1H), 4.04 (s, 3H), 2.27-2.20 (m, 1H), 2.10-1.98 (m, 2H), 1.89-1.65 (m, 3H), 1.49 (s, 9H). MS m/z [M+H]⁺=415.4.

### Intermediate 13b: Tert-butyl (6-((1S,7S)-2,2-difluoro-7-hydroxycycloheptyl)-4-methoxy-5-oxo-6,7-dihydro-5H-pyrrolo[3,4-d]pyrimidin-2-yl)carbamate

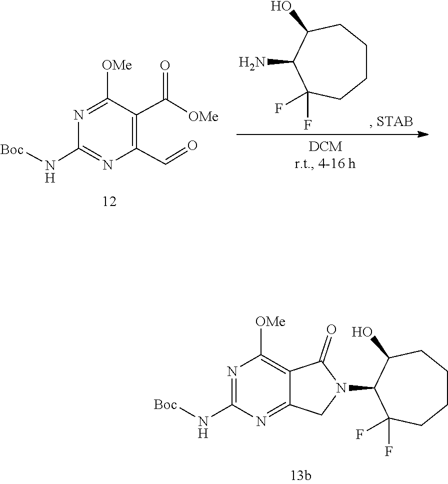 [SMILES: COC(=O)c1c(C=O)nc(NC(=O)OC(C)(C)C)nc1OC ; N[C@H]1[C@@H](O)CCCCC1(F)F ; COc1nc(NC(=O)OC(C)(C)C)nc2c1C(=O)N([C@H]1[C@@H](O)CCCCC1(F)F)C2]

Following general procedure A, compound 13b was obtained from methyl 2-((tert-butoxycarbonyl)amino)-4-formyl-6-methoxypyrimidine-5-carboxylate (1.3 g, 4.2 mmol, Intermediate 12), (1S,2S)-2-amino-3,3-difluoro-cycloheptanol (690 mg, 4.17 mmol) in DCM (30 mL) for 30 min. Followed by STAB (2.65 g, 12.5 mmol) at r.t., overnight. Cyclisation was achieved in THF (30 mL), refluxed, overnight. Column chromatography (silica gel; heptane-EtOAc from 100/0 to 0/100) afforded tert-butyl (6-((1S,7S)-2,2-difluoro-7-hydroxycycloheptyl)-4-methoxy-5-oxo-6,7-dihydro-5H-pyrrolo[3,4-d]pyrimidin-2-yl) carbamate (1.35 g, 73%) this was taken onto the next step without further purification (see synthesis of Compound 50).

### Intermediate 17a: 4-Chloro-6-((1S,7S)-2,2-difluoro-7-hydroxycycloheptyl)-2-(methylthio)-6,7-dihydro-5H-pyrrolo[3,4-d]pyrimidin-5-one

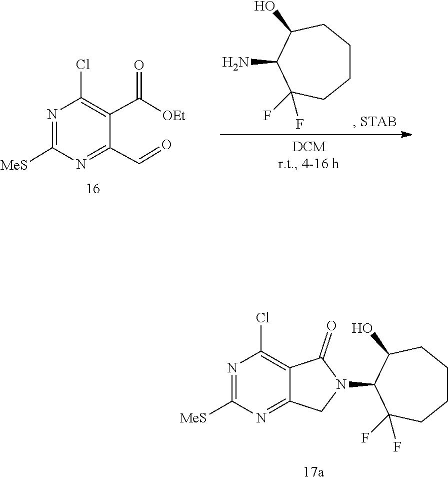 [SMILES: CCOC(=O)c1c(Cl)nc(SC)nc1C=O ; N[C@H]1[C@@H](O)CCCCC1(F)F ; CSc1nc(Cl)c2c(n1)CN([C@H]1[C@@H](O)CCCCC1(F)F)C2=O]

Following general procedure A, compound 17a was obtained from (1S,2S)-2-amino-3,3-difluoro-cycloheptanol (400 mg, 2.42 mmol) and ethyl 4-chloro-6-formyl-2-(methylthio)pyrimidine-5-carboxylate (575 mg, 2.21 mmol, Intermediate 16) in DCM (6 mL) for 1 h. Followed by: STAB (1.40 g, 6.62 mmol) at r.t. for 48 h. Cyclisation was achieved in 1,4-dioxane (5 mL) at reflux, overnight. Column chromatography (silica gel; heptane-EtOAc from 100/0 to 50/50) afforded 4-chloro-6-((1S,7S)-2,2-difluoro-7-hydroxycycloheptyl)-2-(methylthio)-6,7-dihydro-5H-pyrrolo[3,4-d]pyrimidin-5-one in (395 mg, 47%) as a light yellow solid. ¹H NMR (400 MHZ, DMSO-d₆) δ 5.27 (d, J=4.3 Hz, 1H), 4.84 (d, J=19.8 Hz, 1H), 4.71 (dd, J=20.8, 12.1 Hz, 1H), 4.62 (d, 19.8 Hz, 1H), 4.13-4.12 (m, 1H), 2.61 (s, 3H), 2.30-2.14 (m, 2H), 1.95-1.91 (m, 1H), 1.80-1.63 (m, 4H), 1.51-1.43 (m, 1H). MS m/z [M+H]⁺=364.2.

### Intermediate 17b: 4-Chloro-6-((1R,2S)-2-hydroxycyclohexyl)-2-(methylthio)-6,7-dihydro-5H-pyrrolo[3,4-d]pyrimidin-5-one

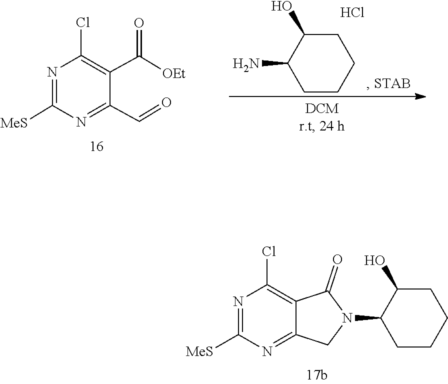 [SMILES: CCOC(=O)c1c(Cl)nc(SC)nc1C=O ; N[C@@H]1CCCC[C@@H]1O ; Cl ; CSc1nc(Cl)c2c(n1)CN([C@@H]1CCCC[C@@H]1O)C2=O]

Following general procedure A, compound 17b was obtained from ethyl 4-chloro-6-formyl-2-(methylthio)pyrimidine-5-carboxylate (4.5 g, 17.3 mmol, Intermediate 16) and (1S, 2R)-2-aminocyclohexanol hydrochloride (2.88 g, 19.0 mmol) in DCM (100 mL) for 0.1 h. Followed by STAB (9.15 g, 43.1 mmol), at r.t. for 24 h. Column chromatography (silica gel; petroleum ether-EtOAc 100/0 to 67/33) to afford 4-chloro-6-((1R,2S)-2-hydroxycyclohexyl)-2-(methylthio)-6,7-dihydro-5H-pyrrolo[3,4-d]pyrimidin-5-one (900 mg, 17%) as a yellow solid. ¹H NMR (400 MHZ, DMSO-d₆) δ 4.78 (d, J=4.0 Hz, 1H), 4.73-4.65 (m, 1H), 4.57-4.49 (m, 1H), 4.05-3.95 (m, 2H), 2.60 (s, 3H), 2.03-1.90 (m, 1H), 1.81-1.66 (m, 2H), 1.61-1.47 (m, 3H), 1.43-1.31 (m, 2H)

### Intermediate 17c: (S)-4-chloro-6-(2,2-difluorocyclohexyl)-2-(methylthio)-6,7-dihydro-5H-pyrrolo[3,4-d]pyrimidin-5-one

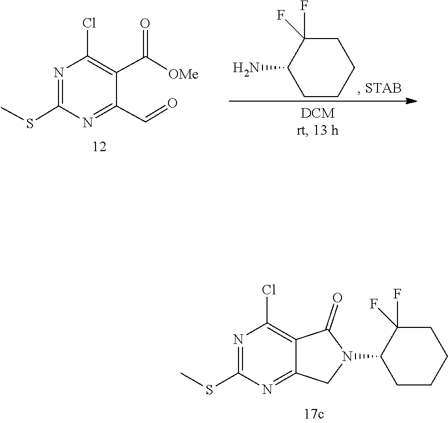 [SMILES: COC(=O)c1c(Cl)nc(SC)nc1C=O ; N[C@H]1CCCCC1(F)F ; CSc1nc(Cl)c2c(n1)CN([C@H]1CCCCC1(F)F)C2=O]

Following general procedure A, compound 17c was obtained from ethyl 4-chloro-6-formyl-2-(methylthio)pyrimidine-5-carboxylate (2.5 g, 9.59 mmol, Intermediate 16) and (1S)-2,2-difluorocyclohexanamine hydrochloride (1.65 g, 9.59 mmol) in DCM (10 mL) for 0.5 h. Followed by STAB (4.06 g, 19.18 mmol) at 40° C. for 12 h. Column chromatography (silica gel; petroleum ether-EtOAc 90/0 to 80/20) to afford(S)-4-chloro-6-(2,2-difluorocyclohexyl)-2-(methylthio)-6,7-dihydro-5H-pyrrolo[3,4-d]pyrimidin-5-one (2.5 g, 63%) as a white solid. ¹H NMR (400 MHZ, DMSO-d₆) δ4.73-4.64 (m, 1H), 4.60-4.45 (m, 2H), 2.60 (s, 3H), 2.20-2.06 (m, 1H), 2.06-1.90 (m, 1H), 1.90-1.74 (m, 4H), 1.62-1.34 (m, 2H).

### Intermediate 18a: 6-((1S,7S)-2,2-difluoro-7-hydroxycycloheptyl)-4-(methylamino)-2-(methylthio)-6,7-dihydro-5H-pyrrolo[3,4-d]pyrimidin-5-one

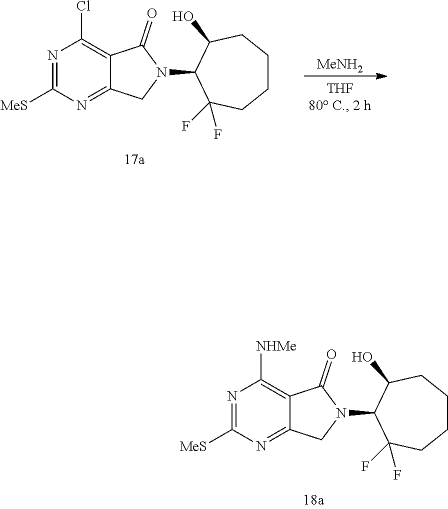 [SMILES: "MeNH2" ; CSc1nc(Cl)c2c(n1)CN([C@H]1[C@@H](O)CCCCC1(F)F)C2=O ; CNc1nc(SC)nc2c1C(=O)N([C@H]1[C@@H](O)CCCCC1(F)F)C2]

To a solution of 4-chloro-6-((1S,7S)-2,2-difluoro-7-hydroxycycloheptyl)-2-(methylthio)-6,7-dihydro-5H-pyrrolo[3,4-d]pyrimidin-5-one (112 mg, 0.31 mmol, Intermediate 17a) in THF (2.5 mL) was added DIPEA (84 μL, 0.62 mmol) followed by methylamine (2M in THF, 230 μL, 0.46 mmol). The reaction was sealed and heated at 80° C. for 2 h. The reaction was cooled to r.t. and diluted with water and DCM, passed through a phase separator and concentrated in vacuo. The reaction was purified by column chromatography (silica gel; heptane-EtOAc from 100/0 to 0/100) to afford 6-((1S,7S)-2,2-difluoro-7-hydroxycycloheptyl)-4-(methylamino)-2-(methylthio)-6,7-dihydro-5H-pyrrolo[3,4-d]pyrimidin-5-one (70 mg, 63%) as a yellow solid. MS m/z [M+H]⁺=359.2.

### Intermediate 22a: 6-((1S,7S)-2,2-Difluoro-7-hydroxycycloheptyl)-2-(methylthio)-4-(1H-pyrazol-1-yl)-6,7-dihydro-5H-pyrrolo[3,4-d]pyrimidin-5-one

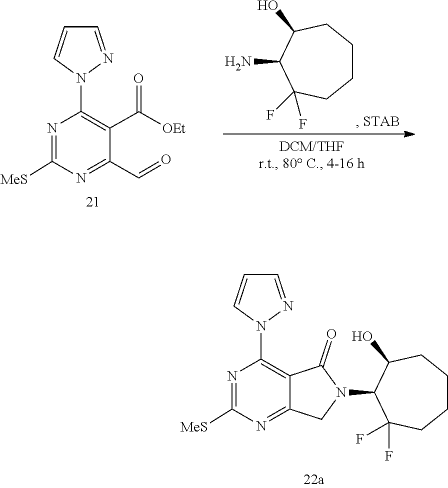 [SMILES: CCOC(=O)c1c(C=O)nc(SC)nc1-n1cccn1 ; N[C@H]1[C@@H](O)CCCCC1(F)F ; CSc1nc2c(c(-n3cccn3)n1)C(=O)N([C@H]1[C@@H](O)CCCCC1(F)F)C2]

Following general procedure A, compound 22a was obtained from ethyl 4-formyl-2-(methylthio)-6-(1H-pyrazol-1-yl)pyrimidine-5-carboxylate (163 mg, 0.56 mmol, Intermediate 21) and (1S,2S)-2-amino-3,3-difluoro-cycloheptanol (92 mg, 0.56 mmol) in DCM (5 mL) for 30 min. Followed by STAB (354 mg, 1.67 mmol), at r.t. overnight. Cyclisation was achieved in THF (5 mL) at reflux for 72 h. Column chromatography (silica gel; heptane-EtOAc from 100/0 to 0/80) afforded 6-((1S,7S)-2,2-difluoro-7-hydroxycycloheptyl)-2-(methylthio)-4-(1H-pyrazol-1-yl)-6,7-dihydro-5H-pyrrolo[3,4-d]pyrimidin-5-one (196 mg, 84%) as a white solid. ¹H NMR (500 MHZ, DMSO-d₆) δ 9.39 (d, J=2.8 Hz, 1H), 7.95 (d, J=1.5 Hz, 1H), 6.66 (dd, J=2.7, 1.6 Hz, 1H), 5.25 (d, J=4.3 Hz, 1H), 4.90 (d, J=19.7 Hz, 1H), 4.78 (dd, J=20.9, 11.4 Hz, 1H), 4.67 (d, J=19.7 Hz, 1H), 4.15-4.14 (m, 1H), 2.66 (s, 3H), 2.29-2.16 (m, 2H), 1.97-1.93 (m, 1H), 1.83-1.77 (m, 1H), 1.74-1.63 (m, 3H), 1.52-1.45 (m, 1H).

### Intermediate: 26a 4-(4-ethoxy-1H-pyrazol-1-yl)-6-((1R,2S)-2-hydroxycyclohexyl)-2-(methylthio)-6,7-dihydro-5H-pyrrolo[3,4-d]pyrimidin-5-one

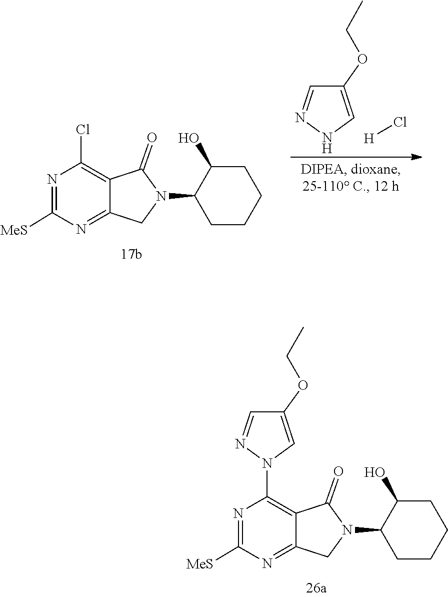

To a mixture of 4-ethoxy-1H-pyrazole hydrochloride (284 mg, 1.91 mmol, Intermediate 25) and 4-chloro-6-((1R,2S)-2-hydroxycyclohexyl)-2-(methylthio)-6,7-dihydro-5H-pyrrolo[3,4-d]pyrimidin-5-one (400 mg, 1.27 mmol, Intermediate 17b) in 1,4-dioxane (10 mL) was added DIPEA (1.11 mL, 6.37 mmol) in one portion at 25° C. under N₂. The mixture was stirred at 110° C. for 12 h. The mixture was filtered and concentrated in vacuo and the residue purified by reverse phase column chromatography (5-95% MeCN in water, 0.1% Formic acid) to afford 4-(4-ethoxy-1H-pyrazol-1-yl)-6-((1R,2S)-2-hydroxycyclohexyl)-2-(methylthio)-6,7-dihydro-5H-pyrrolo[3,4-d]pyrimidin-5-one (300 mg, 57%) as a yellow solid. ¹H NMR (400 MHZ, DMSO-d₆) δ 9.46 (s, 1H), 7.83 (s, 1H), 4.78 (d, J=4.2 Hz, 1H), 4.75-4.67 (m, 1H), 4.60-4.51 (m, 1H), 4.10-3.97 (m, 4H), 2.63 (s, 3H), 2.06-1.94 (m, 1H), 1.83-1.69 (m, 2H), 1.62-1.47 (m, 3H), 1.43-1.31 (m, 5H).

### Intermediate 26b: (S)-6-(2,2-difluorocyclohexyl)-4-(4-ethoxy-1H-pyrazol-1-yl)-2-(methylthio)-6,7-dihydro-5H-pyrrolo[3,4-d]pyrimidin-5-one

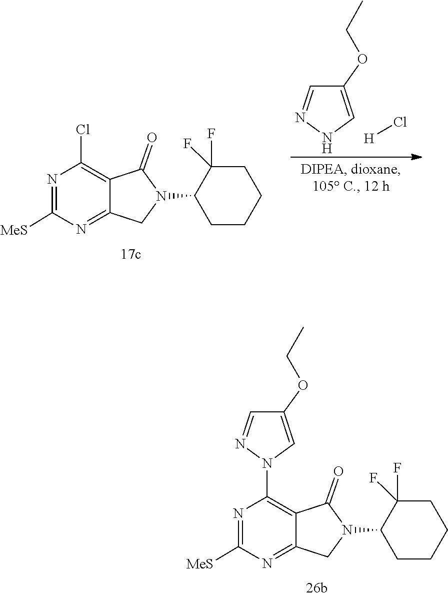

To a mixture of(S)-4-chloro-6-(2,2-difluorocyclohexyl)-2-(methylthio)-6,7-dihydro-5H-pyrrolo[3,4-d]pyrimidin-5-one (400 mg, 1.20 mmol, Intermediate 17c) and 4-ethoxy-1H-pyrazole hydrochloride (267 mg, 1.80 mmol, Intermediate 25) in 1,4-dioxane (3 mL) was added DIPEA (1 mL, 5.99 mmol). The mixture was stirred at 105° C. for 12 h before being cooled to rt, filtered and concentrated in vacuo. The residue was partitioned between water and DCM, the combined organic layers washed with brine, dried over Na₂SO₄, filtered and concentrated in vacuo. The residue was purified by column chromatography (silica gel; petroleum ether-EtOAc 100/0 to 67/33) to afford (S)-6-(2,2-difluorocyclohexyl)-4-(4-ethoxy-1H-pyrazol-1-yl)-2-(methylthio)-6,7-dihydro-5H-pyrrolo[3,4-d]pyrimidin-5-one (450 mg, 92%) as a yellow solid. ¹H NMR (400 MHZ, DMSO-d₆) δ 9.31 (s, 1H), 7.84 (s, 1H), 4.76-4.45 (m, 3H), 4.02 (q, J=6.8 Hz, 2H), 2.63 (s, 3H), 2.13 (br d, J=4.4 Hz, 1H), 2.06-1.92 (m, 1H), 1.91-1.74 (m, 4H), 1.60-1.42 (m, 2H), 1.35 (t, J=6.8 Hz, 3H).

### Synthesis of Amine Building Blocks

### Intermediate 27: 2-aminocycloheptan-1-ol

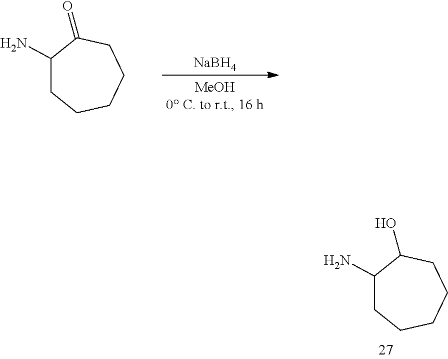 [SMILES: NC1CCCCCC1=O ; "NaBH4" ; NC1CCCCCC1O]

To a solution of 2-aminocycloheptan-1-one hydrochloride (1 g, 6.11 mmol, commercially available), in MeOH (60 mL), cooled to 0° C., was added NaBH₄ (289 mg, 7.64 mmol) portionwise then warmed to r.t. and stirred for 16 h. The reaction was concentrated in vacuo and partitioned between sat. NaHCO_{3(aq)} solution and 3:1 CHCl₃/EtOH, dried over Na₂SO₄ and concentrated in vacuo to afford 2-aminocycloheptan-1-ol (790 mg, quantitative) as an off-white solid. The compound was used without further purification. ¹H NMR (400 MHZ, DMSO-d₆) δ 3.59-3.56 (m, 1H), 2.81-2.78 (m, 1H), 1.68-1.55 (m, 4H), 1.54-1.37 (m, 5H), 1.36-1.27 (m, 2H).

The synthesis of amine intermediate 36 may be prepared according to Intermediate Scheme 6.

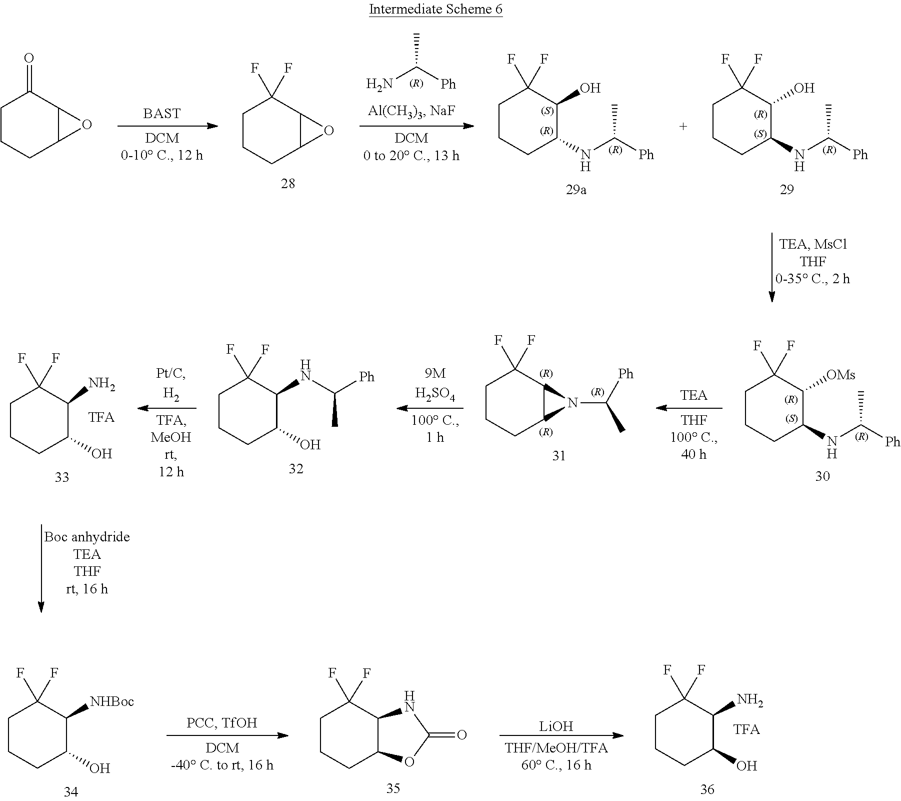

### Intermediate 28: 2,2-difluoro-7-oxabicyclo[4.1.0]heptane

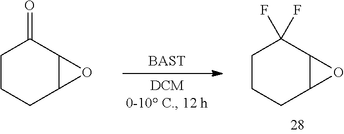 [SMILES: O=C1CCCC2OC12 ; FC1(F)CCCC2OC21]

To a solution of 7-oxabicyclo[4.1.0]heptan-2-one (13 g, 116 mmol, commercially available) in DCM (10 mL) was added BAST (25.4 mL, 116 mmol) at 0° C. dropwise and the mixture was stirred at r.t. for 12 h. The reaction mixture was warmed to 10° C. before water (5 mL) was added drop-wise and then the mixture was passed through a plug of silica gel, the filtrate was concentrated in vacuo at 10° C. to afford 2,2-difluoro-7-oxabicyclo[4.1.0]heptane (15 g) as an off-white solid. The crude product was used in the next step without further purification.

### Intermediate 29 and 29a: (1S,6R)-2,2-difluoro-6-(((R)-1-phenylethyl)amino)cyclohexan-1-ol (25A) and (1R,6S)-2,2-difluoro-6-(((R)-1-phenylethyl)amino)cyclohexan-1-ol (29)

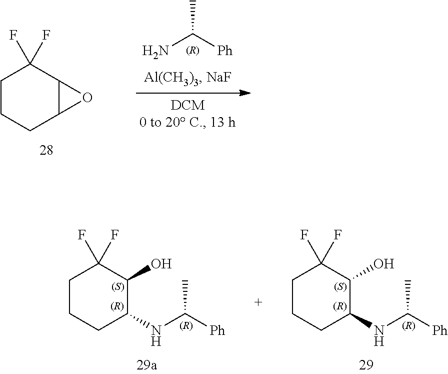

To a mixture of (R)-1-phenylethan-1-amine (16.5 mL, 128 mmol, commercially available) in DCM (200 mL) was added Al(CH₃)₃ (2 M, 61.5 mL, 140.79 mmol) at 0° C. under nitrogen. 2,2-difluoro-7-oxabicyclo[4.1.0]heptane (15 g, 111.84 mmol, Intermediate 28) added and the reaction mixture stirred at 0° C. for 2 h, warmed to r.t. and stirred for 13 h. The reaction was cooled to 10° C., NaF added (12 g) and the reaction stirred for 10 min before ice-water (20 mL) was added dropwise maintaining the temperature around 10° C. The aqueous phase was extracted with EtOAc and the combined organics were washed with brine, dried over Na₂SO₄, filtered and concentrated in vacuo and purified by column chromatography (silica gel; petroleum ether-EtOAc from 90/10 to 50/50) to afford the diastereomers: (1S,6R)-2,2-difluoro-6-(((R)-1-phenylethyl)amino)cyclohexan-1-ol (12 g, 42%) as a white solid. ¹H NMR (400 MHZ, CDCl₃) δ 7.35 (d, J=4.3 Hz, 4H), 7.29-7.24 (m, 1H), 3.93 (q, J=6.6 Hz, 1H), 3.42 (ddd, J=19.8, 9.9, 4.2, Hz, 1H), 2.78-2.68 (m, 1H), 2.22-2.08 (m, 1H), 1.82 (br dd, J=13.3, 1.7 Hz, 1H), 1.74-1.56 (m, 2H), 1.52-1.42 (m, 1H), 1.38 (d, J=6.5 Hz, 3H), 1.06-0.91 (m, 1H).

(1R,6S)-2,2-difluoro-6-(((R)-1-phenylethyl)amino)cyclohexan-1-ol (11 g, 39%) as a white solid. ¹H NMR (400 MHZ, CDCl₃) δ 7.30-7.23 (m, 2H), 7.22-7.13 (m, 3H), 3.86 (q, J=6.6 Hz, 1H), 3.37 (ddd, J=19.9, 10.0, 4.4 Hz, 1H), 2.38-2.27 (m, 1H), 2.08-1.96 (m, 2H), 1.66-1.50 (m, 2H), 1.32-1.19 (m, 4H), 1.07-0.90 (m, 1H).

### Intermediate 30: (1R,6S)-2,2-difluoro-6-(((R)-1-phenylethyl)amino)cyclohexyl methanesulfonate

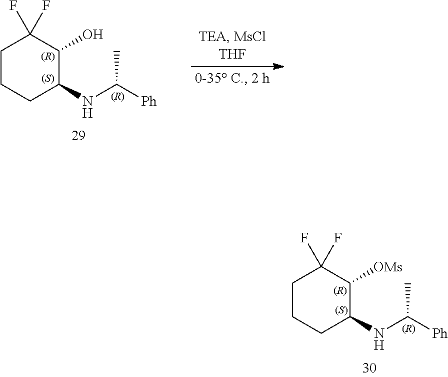

To a solution of (1R,6S)-2,2-difluoro-6-[[(1R)-1-phenylethyl]amino]cyclohexanol (5.9 g, 23.32 mmol, Intermediate 29) in THF (120 mL) cooled to 0° C. was added TEA (3.25 mL, 23.33 mmol) and mesyl chloride (2 mL, 25.66 mmol), the reaction was heated at 35° C. for 16 h, filtered, concentrated in vacuo and purified by column chromatography (silica gel; heptane-EtOAc from 80/20 to 60/40) to afford [(1R,6S)-2,2-difluoro-6-[(1R)-1-phenylethyl]amino]cyclohexyl]methanesulfonate (7.05 g, 91%) as an orange oil. ¹H NMR (400 MHZ, CDCl₃) δ 7.34-7.18 (m, 5H), 4.50 (ddd, J=18.4, 9.8, 4.0 Hz, 1H), 3.90 (q, J=6.6 Hz, 1H), 3.13 (s, 3H), 2.70-2.63 (m, 1H), 2.17-2.07 (m, 2H), 1.76-1.59 (m, 3H), 1.36-1.26 (m, 4H), 1.19-1.08 (m, 1H).

### Intermediate 31: (1S,6S)-2,2-difluoro-7-((R)-1-phenylethyl)-7-azabicyclo[4.1.0]heptane

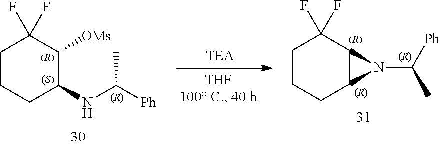

A solution of (1R,6S)-2,2-difluoro-6-(((R)-1-phenylethyl)amino)cyclohexyl methanesulfonate (7.05 g, 42.29 mmol, Intermediate 30) and TEA (6 mL, 42.29 mmol) in 1,4-Dioxane (60 mL) was refluxed for 40 h, cooled to r.t., concentrated in vacuo and purified by column chromatography (silica gel; heptane-EtOAc from 100/0 to 50/50) to afford (1S,6S)-5,5-difluoro-7-[(1R)-1-phenylethyl]-7-azabicyclo[4.1.0]heptane (3.68 g, 73%) as a pale yellow oil. ¹H NMR (400 MHZ, CDCl₃) δ 7.37-7.22 (m, 5H), 2.61 (q, J=6.5 Hz, 1H), 2.05-2.00 (m, 1H), 1.99-1.91 (m, 1H), 1.90-1.87 (m, 2H), 1.84-1.76 (m, 1H), 1.75-1.66 (m, 1H), 1.64-1.50 (m, 1H), 1.40 (d, J=6.5 Hz, 3H). MS m/z [M+H]⁺=238.3.

### Intermediate 32: (1R,2S)-3,3-difluoro-2-(((R)-1-phenylethyl)amino)cyclohexan-1-ol

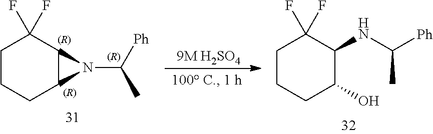

A solution of (1S,6S)-2,2-difluoro-7-((R)-1-phenylethyl)-7-azabicyclo[4.1.0]heptane (3.68 g, 15.51 mmol, Intermediate 31) in H₂SO_{4(aq)} (9N, 45 mL, 698 mmol) was stirred at 100° C. for 1 h. The reaction mixture was basified, to pH 12, with 3M NaOH_{(aq)} and extracted with DCM. The combined organics were dried over Na₂SO₄ and concentrated in vacuo to afford (1R,2S)-3,3-difluoro-2-[[(1R)-1-phenylethyl]amino]cyclohexanol (3.7 g, 93%) as a pale-yellow oil, that was used without further purification. ¹H NMR (400 MHZ, CDCl₃) δ 7.39-7.32 (m, 4H), 7.29-7.25 (m, 1H), 4.29 (q, J=6.4 Hz, 1H), 3.46 (s, 1H), 3.30-3.24 (m, 1H), 2.78-2.69 (m, 1H), 2.22-2.08 (m, 2H), 1.84-1.67 (m, 3H), 1.58-1.39 (m, 1H), 1.35 (d, J=6.4 Hz, 3H). MS m/z [M+H]⁺=256.3.

### Intermediate 33: (1R,2S)-2-amino-3,3-difluorocyclohexan-1-ol 2,2,2-trifluoroacetate

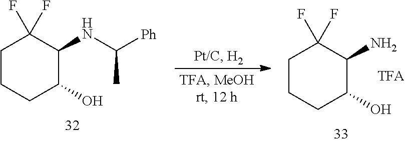 [SMILES: C[C@@H](N[C@H]1[C@H](O)CCCC1(F)F)c1ccccc1 ; N[C@H]1[C@H](O)CCCC1(F)F]

A solution of (1R,2S)-3,3-difluoro-2-[[(1R)-1-phenylethyl]amino]cyclohexanol (4 g, 15.71 mmol, Intermediate 32) in EtOH (250 mL) was hydrogenated (Pt/C, 70° C., 5 atm, 1 mL/min) to afford (1R,2S)-2-amino-3,3-difluoro-cyclohexanol (2.18 g, 92%) as a white solid, that was used without further purification. ¹H NMR (400 MHZ, DMSO-d₆) δ 4.82 (m, 1H), 3.22-3.18 (m, 1H), 2.65 (ddd, J=22.6, 9.3, 4.4 Hz, 1H), 2.02-1.92 (m, 1H), 1.84-1.74 (m, 3H), 1.72-1.61 (m, 2H), 1.38-1.21 (m, 2H).

### Intermediate 34: Tert-butyl ((1S,6R)-2,2-difluoro-6-hydroxycyclohexyl)carbamate

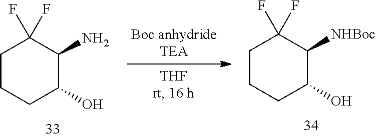 [SMILES: N[C@H]1[C@H](O)CCCC1(F)F ; CC(C)(C)OC(=O)N[C@H]1[C@H](O)CCCC1(F)F]

To a solution of (1R,2S)-2-amino-3,3-difluoro-cyclohexanol (2.18 g, 14.42 mmol, Intermediate 33) in THF (140 mL) was added TEA (4 mL, 28.85 mmol) and di-tert-butyl dicarbonate (3.77 g, 17.31 mmol), the reaction was stirred at r.t. for 16 h, reduced in vacuo and purified by column chromatography (silica gel; heptane-EtOAc from 80/20 to 60/40) to afford tert-butyl ((1S,6R)-2,2-difluoro-6-hydroxycyclohexyl) carbamate (3.06 g, 84%) as a white solid. ¹H NMR (400 MHZ, CDCl₃) δ 4.90 (br s, 1H), 3.81-3.71 (m, 1H), 3.55-3.48 (m, 1H), 2.73 (br s, 1H), 2.23-2.09 (m, 2H), 1.83-1.65 (m, 2H), 1.54-1.36 (m, 11H).

### Intermediate 35: (3aS,7aS)-4,4-difluorohexahydrobenzo[d]oxazol-2 (3H)-one

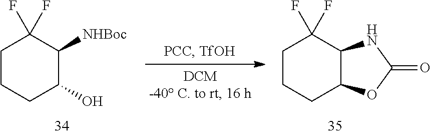 [SMILES: CC(C)(C)OC(=O)N[C@H]1[C@H](O)CCCC1(F)F ; O=C1N[C@H]2[C@H](CCCC2(F)F)O1]

To a solution of tert-butyl ((1S,6R)-2,2-difluoro-6-hydroxycyclohexyl) carbamate (3.06 g, 12.18 mmol, Intermediate 34) in DCM (120 mL) was added PCC (11.8 mL, 146 mmol), the reaction was cooled to −40° C. and trifluoromethanesulfonic anhydride (2.15 mL, 12.79 mmol) was added dropwise maintaining the temperature between −40 and −20° C. Stirred for 1 h, the allowed to warmed to r.t. and stirred for 16 h, diluted with water and passed through a phase separator. The filtrate was concentrated in vacuo and purified by column chromatography (silica gel; heptane-EtOAc from 100/0 to 0/100) to afford (3aS,7aS)-4,4-difluorohexahydrobenzo[d]oxazol-2 (3H)-one (1.95 g, 90%) as a yellow/green solid. ¹H NMR (400 MHZ, DMSO-d₆) δ 8.23 (s, 1H), 4.77-4.72 (m, 1H), 4.00-3.93 (m, 1H), 2.11-1.99 (m, 1H), 1.90-1.70 (m, 2H), 1.66-1.64 (m, 2H).

### Intermediate 36: (1S,2S)-2-amino-3,3-difluorocyclohexan-1-ol 2,2,2-trifluoroacetate

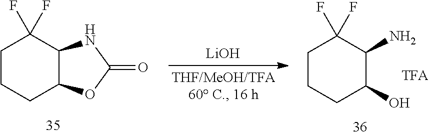 [SMILES: O=C1N[C@H]2[C@H](CCCC2(F)F)O1 ; "LiOH" ; N[C@H]1[C@@H](O)CCCC1(F)F]

To a solution of (3aS,7aS)-4,4-difluorohexahydrobenzo[d]oxazol-2 (3H)-one (1.95 g, 11 mmol, Intermediate 35) in THF (40 mL), MeOH (4 mL) and Water (8 mL) was added lithium hydroxide monohydrate (4.6 g, 110 mmol), the reaction was heated at 60° C. for 48 h. The reaction was filtered through Celite, washed with MeOH and the filtrate reduced in vacuo and partitioned between sat. NaHCO_{3(aq)} solution and 3:1 CHCl₃/IPA solution. The combined organics were passed through a phase separator and concentrated in vacuo before the crude material was dissolved in DCM (20 mL) and TFA (2.5 mL, 33.02 mmol) was added. This was stirred at r.t. for 15 min and reduced in vacuo to afford (1S,2S)-2-amino-3,3-difluorocyclohexan-1-ol 2,2,2-trifluoroacetate (1.75 g, 60%) as a yellow solid. The compound was used in subsequent steps without any further purification. ¹H NMR (400 MHz, DMSO-d₆) δ 8.39 (br s, 3H), 4.09-4.07 (m, 1H), 3.7-3.62 (m, 1H), 2.17-2.07 (m, 1H), 1.96-1.60 (m, 4H), 1.54-1.51 (m, 1H).

The synthesis of amine intermediate 440 may be prepared according to Intermediate Scheme 7.

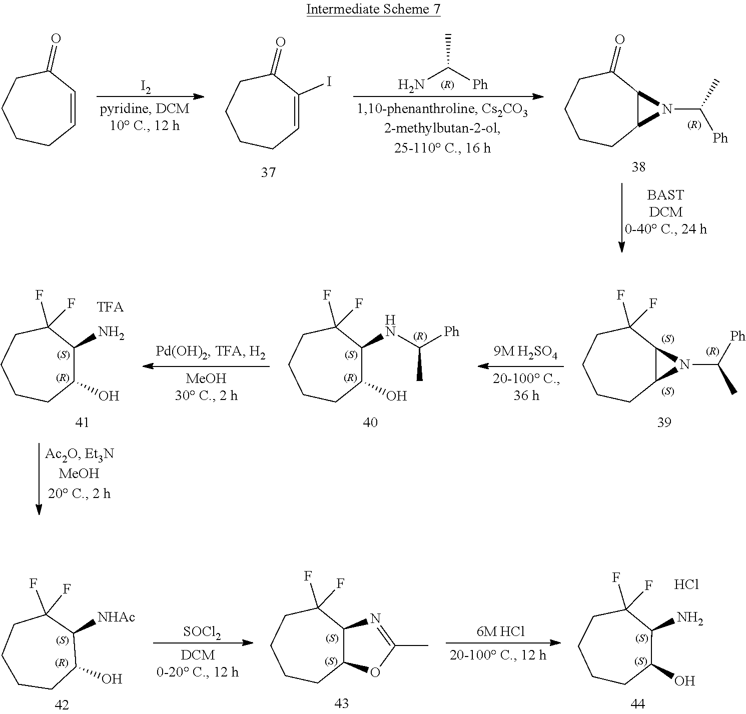

### Intermediate 37: 2-Iodocyclohept-2-en-1-one

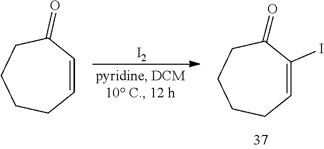 [SMILES: O=C1C=CCCCC1 ; "I2" ; O=C1CCCCC=C1I]

To a solution of cyclohept-2-en-1-one (100 mL, 907 mmol, commercially available) in pyridine (1 L) cooled to 10° C., was added a solution of I₂ (460 g, 1.82 mol) in DCM (1 L) and the reaction mixture stirred at 10° C. for 12 h, filtered and the solid washed with DCM (1 L). The filtrate was diluted with water (1.5 L) and quenched with solid Na₂S₂O₃ (˜165 g) keeping the temperature below 20° C. The organics were washed with 1 N HCl_{(aq)} (1 L), saturated NaHCO_{3(aq)} solution (1 L), dried over Na₂SO₄, filtered and concentrated in vacuo to afford 2-iodocyclohept-2-en-1-one (110 g, 51%) as a yellow oil. The compound was used without further purification. ¹H NMR (400 MHz, CDCl₃) δ 7.64-7.60 (m, 1H), 2.74-2.71 (m, 2H), 2.44-2.42 (m, 2H), 1.85-1.78 (m, 4H).

### Intermediate 38: (1S,7S)-8-((R)-1-phenylethyl)-8-azabicyclo [5.1.0]octan-2-one

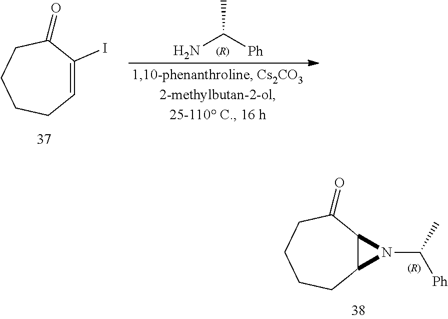

To a solution of 2-iodocyclohept-2-en-1-one (110 g, 466 mmol, Intermediate 37) in 2-methylbutan-2-ol (1 L) was added (R)-1-phenylethan-1-amine (90 mL, 699 mmol) and 1,10-phenanthroline (83.9 g, 466 mmol), the mixture heated to 95° C. before the careful addition of Cs₂CO₃ (227 g, 699 mmol) under nitrogen, the mixture was then stirred at 110° C. for 16 h. The mixture was cooled to r.t. and the mixture was filtered through a pad of silica gel. The filter cake was washed with EtOAc (500 mL) and the filtrate was dried over concentrated in vacuo. The crude product was purified by column chromatography (silica gel; petroleum ether-EtOAc from 100/0 to 5/95) to afford (1S,7S)-8-((R)-1-phenylethyl)-8-azabicyclo [5.1.0]octan-2-one (65 g, 61%) as a yellow oil. ¹H NMR (400 MHZ, CDCl₃) δ 7.38-7.25 (m, 5H), 2.96-2.93 (m, 1H), 2.83-2.83 (m, 1H), 2.66-2.62 (m, 1H), 2.18-2.15 (m, 2H), 1.84-1.80 (m, 2H), 1.72-1.63 (m, 3H), 1.42-1.39 (m, 3H), 0.98-0.94 (m, 1H).

### Intermediate 39: (1S,7S)-2,2-difluoro-8-((R)-1-phenylethyl)-8-azabicyclo[5.1.0]octane

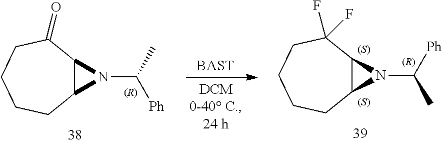

To a solution of (1S,7S)-8-((R)-1-phenylethyl)-8-azabicyclo [5.1.0]octan-2-one (65 g, 283 mmol, Intermediate 38) in DCM (650 mL) cooled to 0° C. was added BAST (248 mL, 1.13 mol) drop-wise, the reaction was stirred at 40° C. for 24 h, cooled to r.t. and poured onto ice water (1 L) slowly and washed with brine. The combined organics were dried over Na₂SO₄, filtered and concentrated in vacuo and purified by column chromatography (silica gel; petroleum ether-EtOAc from 100/0 to 5/95) to afford (1S,7S)-8-((R)-1-phenylethyl)-8-azabicyclo [5.1.0]octan-2-one (21 g, 29%) as a yellow oil. ¹H NMR (400 MHZ, CDCl₃) δ 7.36-7.31 (m, 4H), 7.27-7.23 (m, 1H), 2.70-2.67 (m, 1H), 2.28-2.06 (m, 1H), 1.92-1.72 m, 4H), 1.62-1.59 (m, 4H), 1.40-1.38 (m, 3H), 1.17-1.14 (m, 1H).

### Intermediate 40: (1R,2S)-3,3-difluoro-2-(((R)-1-phenylethyl)amino)cycloheptan-1-ol

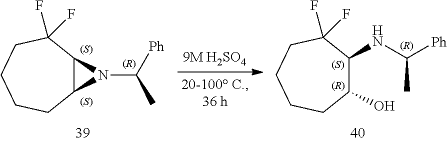

H₂SO_{4(aq)}, (9 M, 105 mL) was added to (1S,7S)-2,2-difluoro-8-((R)-1-phenylethyl)-8-azabicyclo[5.1.0]octane (21 g, 83.5 mmol, Intermediate 39) at 20° C. The mixture was stirred at 100° C. for 36 h. The mixture was adjusted to pH 7 with NaOH_{(aq)} (8 M). The resulting mixture was filtered, washed with EtOAc and the combined organics were washed with brine, dried over Na₂SO₄ and concentrated in vacuo. The crude product was purified by column chromatography (Silica gel; petroleum ether-EtOAc from 4/96 to 20/80) to afford (1R,2S)-3,3-difluoro-2-(((R)-1-phenylethyl)amino)cycloheptan-1-ol (25 g, 75%) as a yellow oil. ¹H NMR (400 MHZ, CDCl₃) δ 7.29-7.18 (m, 5H), 4.14-4.19 (m, 1H), 3.23-3.20 (m, 1H), 2.84-2.79 (m, 1H), 2.19-2.11 (m, 2H), 1.75-1.74 (m, 2H), 1.75-1.74 (m, 1H), 1.48-1.45 (m, 3H), 1.29-1.28 (m, 3H).

### Intermediate 41: (1R,2S)-2-amino-3,3-difluorocycloheptan-1-ol 2,2,2-trifluoroacetate

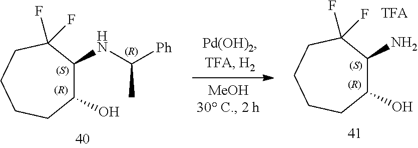

To a solution of (1R,2S)-3,3-difluoro-2-(((R)-1-phenylethyl)amino)cycloheptan-1-ol (25 g, 92.8 mmol, Intermediate 40) in MeOH (120 mL) was added Pd(OH)₂ (2.00 g, 14.2 mmol) and TFA (6.87 mL, 92.8 mmol) under nitrogen. The suspension was degassed under vacuum and purged with H₂ several times. The mixture was stirred under H₂ (15 psi) at 30° C. for 2 h then filtered through celite and the filtrate was concentrated in vacuo. The compound was used without further purification. (1R,2S)-2-amino-3,3-difluorocycloheptan-1-ol 2,2,2-trifluoroacetate (24 g, 93%) was obtained as a yellow oil. ¹H NMR (400 MHZ, CD₃OD) δ 3.73-3.70 (m, 1H), 3.59-3.51 (m, 1H), 2.32-2.03 (m, 3H), 1.81-1.67 (m, 5H).

### Intermediate 42: N-((1S,7R)-2,2-difluoro-7-hydroxycycloheptyl) acetamide

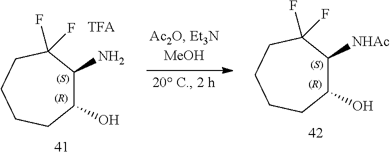

TEA (18 mL, 129 mmol) and acetyl acetate (9.66 mL, 103 mmol) were added dropwise to a cooled (0° C.) solution of (1R,2S)-2-amino-3,3-difluorocycloheptan-1-ol 2,2,2-trifluoroacetate (24 g, 86 mmol, Intermediate 41) in MeOH (250 mL). The mixture was stirred at 20° C. for 16 h, concentrated in vacuo and purified by column chromatography (silica gel; DCM-MeOH from 100/0 to 90/10) to afford N-((1S,7R)-2,2-difluoro-7-hydroxycycloheptyl) acetamide (14 g, 79%) as a white solid. ¹H NMR (400 MHZ, CD₃OD) δ 4.28-4.19 (m, 1H), 3.62-3.57 (m, 1H), 2.11-2.08 (m, 2H), 1.82 (s, 3H), 1.82-1.79 (m, 1H), 1.80-1.79 (m, 2H), 1.68-1.66 (m, 2H), 1.66-1.57 (m, 1H).

### Intermediate 43: (3aS,8aS)-4,4-difluoro-2-methyl-3a,5,6,7,8,8a-hexahydro-4H-cyclohepta[d]oxazole

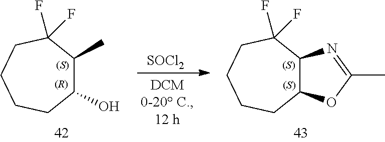

SOCl₂ (9.80 mL, 135 mmol) was added to a solution of N-((1S,7R)-2,2-difluoro-7-hydroxycycloheptyl) acetamide (14 g, 67.5 mmol, Intermediate 42) in DCM (140 mL), cooled to 0° C. The mixture was stirred at 20° C. for 12 h and concentrated in vacuo to afford (3aS,8aS)-4,4-difluoro-2-methyl-3a,5,6,7,8,8a-hexahydro-4H-cyclohepta[d]oxazole (14.8 g, crude) as a white solid. The compound was used without further purification. ¹H NMR (400 MHZ, CDCl₃) δ 5.71-5.70 (m, 1H), 5.48-5.40 (m, 1H), 2.64 (s, 3H), 2.26-2.19 (m, 3H), 2.04-2.01 (m, 1H), 1.74-1.56 (m, 4H).

### Intermediate 44: (1S,2S)-2-amino-3,3-difluorocycloheptan-1-ol hydrochloride

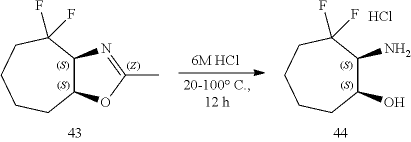

To a solution of (3aS,8aS)-4,4-difluoro-2-methyl-3a,5,6,7,8,8a-hexahydro-4H-cyclohepta[d]oxazole (14 g, 74 mmol, Intermediate 43) in H₂O (140 mL) was added HCl_{(aq)} (12 M, 140 mL) and the reaction was stirred at 100° C. for 12 h. The mixture was concentrated in vacuo to afford (1S,2S)-2-amino-3,3-difluorocycloheptan-1-ol hydrochloride (12.8 g, 86%) as a light yellow solid. The compound was used in subsequent steps without purification. ¹H NMR (400 MHZ, CD₃OD) δ 4.19-4.17 (m, 1H), 3.84-3.79 (m, 1H), 2.29-2.22 (m, 2H), 1.99-1.97 (m, 1H), 1.80-1.77 (m, 3H), 1.64-1.58 (m, 2H). MS m/z [M+H]⁺=166.1

### General Procedure B: Oxidation of SMe and Displacement with Ammonia

To a solution of substituted 2-methyl thiopyrimidine (1 eq) in MeCN (0.04-0.6 M) or 1,4-dioxane (0.12 M) was added oxone (1-1.1 eq) as a suspension in Water (0.5 mL) and the reaction stirred for a suitable time, as detailed in the individual experimental. The reaction was diluted with DCM and water, the organic layer collected through a hydrophobic frit and concentrated in vacuo. NH₃ (0.5M in dioxane, in excess) was added, the reaction sealed and heated at 80° C. for a suitable time 4 to 16 h, cooled to r.t., concentrated in vacuo and purified.

### General Procedure C: Boc Deprotection

To a solution of the Boc-protected amine in DCM (0.12-0.5 M) was added TFA (0.33 mL-4 mL) and stirred at r.t. for a suitable time, 2-16 h. The volatiles were removed in vacuo and the residue subject to purification conditions.

### Compound 45: 2-Amino-4-ethoxy-6-[(1R,2S)-2-hydroxycyclohexyl]-7H-pyrrolo[3,4-d]pyrimidin-5-one

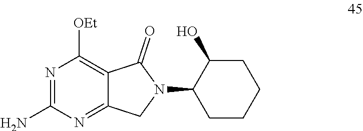 [SMILES: CCOc1nc(N)nc2c1C(=O)N([C@@H]1CCCC[C@@H]1O)C2]

Following general procedure B, Compound 45 was obtained from 4-ethoxy-6-((1R,2S)-2-hydroxycyclohexyl)-2-(methylthio)-6,7-dihydro-5H-pyrrolo[3,4-d]pyrimidin-5-one (1.53 g, 4.63 mmol, Intermediate 6a) and oxone (2.79 g, 4.44 mmol), in 1,4-dioxane (10 mL) for 1 h. Followed by NH₃ (0.5M in 1,4-dioxane, 12 mL) at 80° C. for 4 h. Purification by prep-HPLC afforded 2-amino-4-ethoxy-6-[(1R,2S)-2-hydroxycyclohexyl]-7H-pyrrolo[3,4-d]pyrimidin-5-one (865 mg, 67%) as a white solid. ¹H NMR (400 MHz, CDCl₃) δ 5.19 (br s, 2H), 4.53-4.48 (m, 3H), 4.25 (br s, 1H), 4.2 (d, J=18.7 Hz, 1H), 3.95-3.91 (m, 1H), 3.75-3.68 (m, 1H), 2.90 (d, J=3.44 Hz, 1H), 2.12 (ddd, J=25.2, 12.6, 3.5 Hz, 1H), 1.88-1.82 (m, 2H), 1.74-1.54 (m, 2H), 1.50-1.35 (m, 5H), 1.27-1.22 (m, 1H). MS m/z [M+H]⁺=257.2.Prep-HPLC conditions: Column: Waters XBridge C18 columns, 19×100 mm, 5 μm particle size, Mobile phase: A-0.1% ammonium hydroxide (aq) B-acetonitrile, Method (% of B): linear 5-95%, Flow: 24 mL/min, Temperature: ambient.

### Compound 46: 2-Amino-6-(2-hydroxycycloheptyl)-4-ethoxy-6,7-dihydro-5H-pyrrolo[3,4-d]pyrimidin-5-one

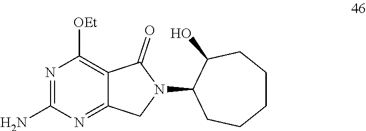 [SMILES: CCOc1nc(N)nc2c1C(=O)N([C@@H]1CCCCC[C@@H]1O)C2]

Following general procedure B, Compound 46 was obtained from 6-(2-hydroxycycloheptyl)-4-ethoxy-2-(methylthio)-6,7-dihydro-5H-pyrrolo[3,4-d]pyrimidin-5-one (608 mg, 1.8 mmol, Intermediate 6b) and oxone (1.33 g, 2.16 mmol) in MeCN (30 mL) for 16 h. Followed by NH₃ (0.5 M in 1,4-dioxane, 5 mL) at r.t., overnight. Column chromatography (silica gel; DCM-MeOH from 100/0 to 90/10). Chiral Separation by SFC: Column: ChiralpakIC (30 mm×250 mm) 5μ, Isocratic method: CO₂:EtOH, 80:20 during 16 min, Flow: 100 mL/min, Rₜ=13.88 min. ¹H NMR (400 MHZ, CDCl₃) δ 5.22 (br s, 2H), 4.51 (q, J=7.1 Hz, 2H), 4.38 (d, J=18.6 Hz, 1H), 4.31-4.29 (m, 1H), 4.22 (d, J=18.6 Hz, 1H), 3.98-3.71 (m, 1H), 3.84-3.80 (m, 1H), 2.34-2.24 (m, 1H), 1.95-1.88 (m, 1H), 1.83-1.72 (m, 2H), 1.71-1.51 (m, 6H), 1.44 (t, J=7.1 Hz, 3H).

### Compound 47: 2-Amino-6-((1S,6S)-2,2-difluoro-6-hydroxycyclohexyl)-4-ethoxy-6,7-dihydro-5H-pyrrolo[3,4-d]pyrimidin-5-one

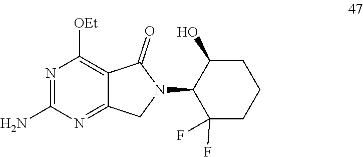 [SMILES: CCOc1nc(N)nc2c1C(=O)N([C@H]1[C@@H](O)CCCC1(F)F)C2]

Following general procedure B, Compound 47 was obtained from 6-((1S,6S)-2,2-difluoro-6-hydroxycyclohexyl)-4-ethoxy-2-(methylthio)-6,7-dihydro-5H-pyrrolo[3,4-d]pyrimidin-5-one (75 mg, 0.21 mmol, Intermediate 6c) and oxone (154 mg, 0.25 mmol) in MeCN (5 mL), overnight. Followed by; NH₃ (0.5 M in 1,4-dioxane, 2.5 mL) at r.t. for 2 h. Purification by prep-HPLC afforded 2-amino-6-((1S,6S)-2,2-difluoro-6-hydroxycyclohexyl)-4-ethoxy-6,7-dihydro-5H-pyrrolo[3,4-d]pyrimidin-5-one (40 mg, 55%) as a white solid. ¹H NMR (500 MHZ, DMSO-d₆) δ 7.26 (br s, 2H), 5.00 (d, J=4.5 Hz, 1H), 4.75 (d, J=19.0 Hz, 1H), 4.48-4.40 (m, 1H), 4.43 (q, J=7.1 Hz, 2H), 4.35 (d, J=19.0 Hz, 1H), 4.07 (br s, 1H), 3.93 (s, 3H), 2.14-1.84 (m, 3H), 1.76-1.65 (m, 2H), 1.60-1.54 (m, 1H), 1.33 (t, J=7.1 Hz, 3H). MS m/z [M+H]⁺=329.14. Prep-HPLC conditions: Column: Waters XBridge C18 columns, 19×100 mm, 5 um particle size, Mobile phase: A-0.1% ammonium hydroxide (aq) B-acetonitrile, Method (% of B): linear 5-95%, Flow: 24 mL/min, Temperature: ambient.

### Compound 48: 2-Amino-6-(2-hydroxycycloheptyl)-4-methoxy-6,7-dihydro-5H-pyrrolo[3,4-d]pyrimidin-5-one

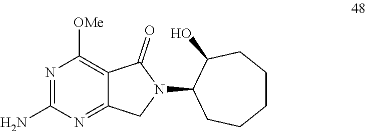 [SMILES: COc1nc(N)nc2c1C(=O)N([C@@H]1CCCCC[C@@H]1O)C2]

Following general procedure A and then C, Compound 48 was obtained from methyl 2-((tert-butoxycarbonyl)amino-4-formyl-6-methoxypyrimidine-5-carboxylate (3.76 g, 12.07 mmol, Intermediate 12) in DCM (60 mL) and 2-aminocycloheptan-1-ol (1.56 g, 12.07 mmol, Intermediate 14) for 1 h, followed by the addition of STAB (7.67 g, 36.22 mmol). Cyclisation was achieved in DCM (40 mL) and refluxed for 12 h to afford tert-butyl (6-(2-hydroxycycloheptyl)-4-methoxy-5-oxo-6,7-dihydro-5H-pyrrolo[3,4-d]pyrimidin-2-yl) carbamate. Following general procedure C, tert-butyl (6-(2-hydroxycycloheptyl)-4-methoxy-5-oxo-6,7-dihydro-5H-pyrrolo[3,4-d]pyrimidin-2-yl) carbamate in DCM (30 mL) and TFA (1 mL) were stirred overnight. Purification was achieved by SCX. Impure fractions were dissolved in MeCN, NaOH_{(aq)} (2M, 1.5 mL) was added and the reaction stirred at r.t. for 4 h. The reaction was concentrated in vacuo and purified by SCX. Both batches were combined and separated by Chiral SFC: Column: ChiralpakIC (30 mm×250 mm) 5μ, Isocratic method: CO₂:EtOH, 70:30 during 16 min, Flow: 100 mL/min, Rₜ=8.53 min. ¹H NMR (400 MHZ, DMSO-d₆) δ 7.21 (br s, 2H), 4.74 (d, J=4.4 Hz, 1H), 4.41 (d, J=18.8 Hz, 1H), 4.20 (d, J=18.8 Hz, 1H), 4.21 (d, J=18.8 Hz, 1H), 4.02 (d, J=11.2 Hz, 1H), 3.93 (br s, 1H), 3.89 (s, 3H), 2.04-1.94 (m, 1H), 1.73-1.54 (m, 9H).

### Compound 49: 2-Amino-6-((1S,6S)-2,2-difluoro-6-hydroxycyclohexyl)-4-methoxy-6,7-dihydro-5H-pyrrolo[3,4-d]pyrimidin-5-one

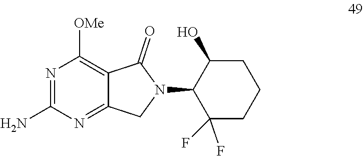 [SMILES: COc1nc(N)nc2c1C(=O)N([C@H]1[C@@H](O)CCCC1(F)F)C2]

Following general procedure C, Compound 49 was obtained from tert-butyl (6-((1S,6S)-2,2-difluoro-6-hydroxycyclohexyl)-4-methoxy-5-oxo-6,7-dihydro-5H-pyrrolo[3,4-d]pyrimidin-2-yl)carbamate (180 mg, 0.43 mmol, Intermediate 13a) and TFA (0.33 mL) in DCM (8 mL) for 16 h. Purification by prep-HPLC afforded 2-amino-6-((1S,6S)-2,2-difluoro-6-hydroxycyclohexyl)-4-methoxy-6,7-dihydro-5H-pyrrolo[3,4-d]pyrimidin-5-one (105 mg, 75%), as a white solid. ¹H NMR (500 MHz, DMSO-d₆) δ 7.30 (s, 2H), 5.00 (d, J=4.7 Hz, 1H), 4.66 (d, J=19.0 Hz, 1H), 4.46 (dt, J=27, 4 Hz, 1H), 4.36 (d, J=19.0 Hz, 1H), 4.07 (br s, 1H), 3.93 (s, 3H), 2.13-1.84 (m, 3H), 1.75-1.66 (m, 2H), 1.60-1.52 (m, 1H). MS m/z [M+H]⁺=315.13.Prep-HPLC conditions: Column: Waters XBridge C18 columns, 19×100 mm, 5 um particle size, Mobile phase: A-0.1% Formic acid (aq) B-acetonitrile, Method (% of B): linear 5-95%, Flow: 24 mL/min, Temperature: ambient.

### Compound 50: 2-Amino-6-((1S,7S)-2,2-difluoro-7-hydroxycycloheptyl)-4-methoxy-6,7-dihydro-5H-pyrrolo[3,4-d]pyrimidin-5-one

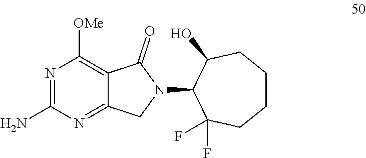 [SMILES: COc1nc(N)nc2c1C(=O)N([C@H]1[C@@H](O)CCCCC1(F)F)C2]

Following general procedure C, Compound 50 was obtained from tert-butyl (6-((1S,7S)-2,2-difluoro-7-hydroxycycloheptyl)-4-methoxy-5-oxo-6,7-dihydro-5H-pyrrolo[3,4-d]pyrimidin-2-yl)carbamate (1.35 g, 3.26 mmol, Intermediate 13b) in TFA (1 mL) for 2 h. Purification by SCX afforded 2-amino-6-((1S,7S)-2,2-difluoro-7-hydroxycycloheptyl)-4-methoxy-6,7-dihydro-5H-pyrrolo[3,4-d]pyrimidin-5-one (644 mg, 46%) as an off white solid. ¹H NMR (400 MHZ, CDCl₃) δ 5.50 (br s, 1H), 5.30 (br s, 2H), 4.64 (d, J=18.4 Hz, 1H), 4.43-4.40 (m, 1H), 4.31 (d, J=18.4 Hz, 1H), 4.05 (s, 3H), 3.91-3.84 (m, 1H), 2.55-2.20 (m, 3H), 1.91-1.74 (m, 3H), 1.63-1.55 (m, 1H), 1.48-1.38 (m, 1H). MS m/z [M+H]⁺=329.2.

### Compound 51: 2-Amino-6-((1S,7S)-2,2-difluoro-7-hydroxycycloheptyl)-4-(methylamino)-6,7-dihydro-5H-pyrrolo[3,4-d]pyrimidin-5-one formate

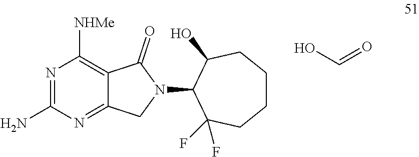 [SMILES: CNc1nc(N)nc2c1C(=O)N([C@H]1[C@@H](O)CCCCC1(F)F)C2 ; O=CO]

Following general procedure B, Compound 51 was obtained from 6-((1S,7S)-2,2-difluoro-7-hydroxycycloheptyl)-4-(methylamino)-2-(methylthio)-6,7-dihydro-5H-pyrrolo[3,4-d]pyrimidin-5-one (70 mg, 0.19 mmol, Intermediate 18) in MeCN (5 mL) and oxone (126 mg, 0.21 mmol) in water (0.5 mL) overnight. Followed by: NH₃ (0.5M in 1,4-dioxane, 3 mL) at 50° C. for 2 h. Purification by prep-HPLC afforded 2-amino-6-((1S,7S)-2,2-difluoro-7-hydroxycycloheptyl)-4-(methylamino)-6,7-dihydro-5H-pyrrolo[3,4-d]pyrimidin-5-one formate (2 mg, 3%) as a white solid. ¹H NMR (500 MHZ, DMSO-d₆) δ 6.77 (m, 3H), 5.24 (s, 1H), 4.55-4.44 (m, 2H), 4.27 (d, J=18.4 Hz, 1H), 4.03 (s, 1H), 2.89 (d, J=4.83 Hz, 3H), 2.24-2.11 (m, 2H), 1.93-1.89 (m, 1H), 1.78-1.73 (m, 1H), 1.71-1.61 (m, 3H), 1.48-1.41 (m, 1H). MS m/z [M+H]⁺=328.4. Prep-HPLC conditions: Column: Waters XBridge C18 columns, 19×100 mm, 5 um particle size, Mobile phase: A-0.1% Formic acid (aq) B-acetonitrile, Method (% of B): linear 5-95%, Flow: 24 mL/min, temperature: ambient.

### Compound 52: 2-Amino-6-((1S,7S)-2,2-difluoro-7-hydroxycycloheptyl)-4-(1H-pyrazol-1-yl)-6,7-dihydro-5H-pyrrolo[3,4-d]pyrimidin-5-one

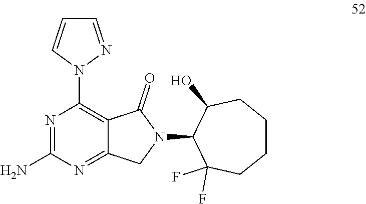 [SMILES: Nc1nc2c(c(-n3cccn3)n1)C(=O)N([C@H]1[C@@H](O)CCCCC1(F)F)C2]

Following general procedure B, Compound 52 was obtained from 6-((1S,7S)-2,2-difluoro-7-hydroxycycloheptyl)-2-(methylthio)-4-(1H-pyrazol-1-yl)-6,7-dihydro-5H-pyrrolo[3,4-d]pyrimidin-5-one (186 mg, 0.47 mmol, Intermediate 22a) in MeCN (6 mL) and oxone (396 mg, 0.64 mmol) in water (2 mL) at r.t. for 2 h. NH₃ (0.5M in 1,4-dionxane, 3 mL) at r.t. for 4 h. Column chromatography (0-10% methanolic ammonia in DCM), followed by reverse phase column chromatography (5-95% MeCN in water, 0.1% Formic acid) afforded 2-amino-6-((1S,7S)-2,2-difluoro-7-hydroxycycloheptyl)-4-(1H-pyrazol-1-yl)-6,7-dihydro-5H-pyrrolo[3,4-d]pyrimidin-5-one (10 mg, 6%) as a white solid. ¹H NMR (400 MHZ, DMSO-d₆) δ 9.31 (d, J=2.7 Hz, 1H), 7.83 (d, J=1.6 Hz, 1H), 7.77 (br s, 1H), 7.67 (br s, 1H), 6.57 (dd, J=2.7, 1.6 Hz, 1H), 5.24 (d, J=4.3 Hz, 1H), 4.75-4.66 (m, 2H), 4.47 (d, J=18.8 Hz, 1H), 4.11-4.09 (m, 1H), 2.27-2.14 (m, 2H), 1.95-1.90 (m, 1H), 1.80-1.72 (m, 1H), 1.70-1.61 (m, 3H), 1.5-1.42 (m, 1H).

### Compound: 53 2-Amino-4-(4-ethoxy-1H-pyrazol-1-yl)-6-((1R,2S)-2-hydroxycyclohexyl)-6,7-dihydro-5H-pyrrolo[3,4-d]pyrimidin-5-one

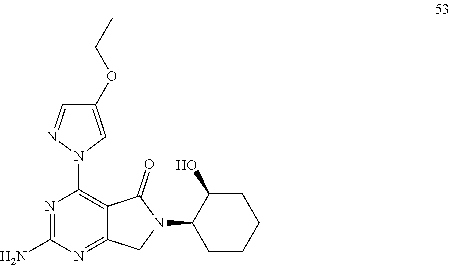 [SMILES: CCOc1cnn(-c2nc(N)nc3c2C(=O)N([C@@H]2CCCC[C@@H]2O)C3)c1]

Following general procedure B, Compound 53 was obtained from 4-(4-ethoxy-1H-pyrazol-1-yl)-6-((1R,2S)-2-hydroxycyclohexyl)-2-(methylthio)-6,7-dihydro-5H-pyrrolo[3,4-d]pyrimidin-5-one (250 mg, 0.642 mmol, Intermediate 26a) and oxone (987 mg, 1.60 mmol) in MeCN (12 mL) for 0.5 h. Followed by NH₃ (0.5 M in 1,4-dioxane, 5 mL) at 25° C. for 1 h. Purification by prep-HPLC afforded 2-amino-4-(4-ethoxy-1H-pyrazol-1-yl)-6-((1R,2S)-2-hydroxycyclohexyl)-6,7-dihydro-5H-pyrrolo[3,4-d]pyrimidin-5-one (16.4 mg, 0.041 mmol, 9%). ¹H NMR (400 MHZ, methanol-d₄) δ 9.50 (s, 1H), 7.66 (s, 1H), 4.68 (d, J=19.2 Hz, 1H), 4.40 (d, J=19.2 Hz, 1H), 4.18-4.11 (m, 2H), 4.06 (q, J=7.2 Hz, 2H), 2.14-2.04 (m, 1H), 1.95-1.81 (m, 2H), 1.72-1.61 (m, 3H), 1.53-1.45 (m, 2H), 1.41 (t, J=7.2 Hz, 3H). MS m/z [M+H]⁺=359.2. Prep-HPLC conditions: Column: Waters XBridge C18 columns, 150×25 mm, 5 um particle size, Mobile phase: A-0.1% NH₄HCO_{3 (aq)} B-acetonitrile, Method (% of B): linear 24-54%, Flow: 24 mL/min, Temperature: ambient.

### Compound 54 (S)-2-amino-6-(2,2-difluorocyclohexyl)-4-(4-ethoxy-1H-pyrazol-1-yl)-6,7-dihydro-5H-pyrrolo[3,4-d]pyrimidin-5-one

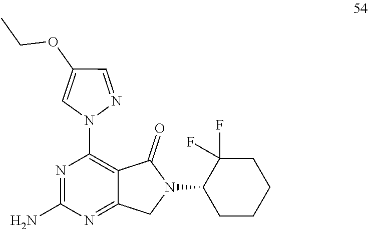 [SMILES: CCOc1cnn(-c2nc(N)nc3c2C(=O)N([C@H]2CCCCC2(F)F)C3)c1]

Following general procedure B, Compound 54 was obtained from(S)-6-(2,2-difluorocyclohexyl)-4-(4-ethoxy-1H-pyrazol-1-yl)-2-(methylthio)-6,7-dihydro-5H-pyrrolo[3,4-d]pyrimidin-5-one (400 mg, 0.98 mmol, Intermediate 26b) in water (1 mL) and THF (5 mL) 0.5 h. Followed by by NH₃ (0.5 M in 1,4-dioxane, 5 mL) 20° C. for 0.2 h Column chromatography (silica gel; petroleum ether-EtOAc 100/0 to 40/60) afforded(S)-2-amino-6-(2,2-difluorocyclohexyl)-4-(4-ethoxy-1H-pyrazol-1-yl)-6,7-dihydro-5H-pyrrolo[3,4-d]pyrimidin-5-one (52.46 mg, 16%) as a white solid. ¹H NMR (400 MHZ, DMSO-d₆) δ 9.29 (s, 1H), 7.77-7.50 (m, 3H), 4.61-4.46 (m, 2H), 4.34-4.27 (m, 1H), 3.99 (q, J=6.8 Hz, 2H), 2.18-2.07 (m, 1H), 2.03-1.83 (m, 2H), 1.79 (br d, J=8.8 Hz, 3H), 1.58-1.41 (m, 2H), 1.34 (t, J=6.8 Hz, 3H). MS m/z [M+H]⁺=379.2.

### Biological Activity

Assay 1 Mycobacterium tuberculosis Lysine tRNA Synthetase Inhibition Assay
Protein Expression and Purification Method: For his-TEV-MtbKRS

The gene encoding Mycobacterium tuberculosis lysyl-tRNA-synthetase (Accession number CCP46421) was codon optimized and synthesized by Genscript, with additional NdeI and XhoI restriction sites added at the termini of the gene. The gene was subsequently digested out the provided pUC57 vector and ligated into a modified pET15B vector encoding an N-terminal Hexa-His tag with an additional Tobacco Etch Virus (TEV) cleavage site between the protein and the tag to allow increased soluble expression, ease of affinity capture and subsequent cleavage during purification if required. All plasmids were sequenced to confirm. The encoded sequence is provided hereinafter:

```
  (SEQ ID NO: 1)
His Tag-MGSSHHHHHHGSS
  (SEQ ID NO: 2)
TEV Clevage site-GENLYFQGH
  (SEQ ID NO: 3)
MtKRS-MSAADTAEDLPEQFRIRRDKRARLLAQGRDPYPVAVPRTHTLAE
VRAAHPDLPIDTATEDIVGVAGRVIFARNSGKLCFATLQDGDGTQLQVMI
SLDKVGQAALDAWKADVDLGDIVYVHGAVISSRRGELSVLADCWRIAAKS
LRPLPVAHKEMSEESRVRQRYVDLIVRPEARAVARLRIAVVRAIRTALQR
RGFLEVETPVLQTLAGGAAARPFATHSNALDIDLYLRIAPELFLKRCIVG
GFDKVFELNRVFRNEGADSTHSPEFSMLETYQTYGTYDDSAVVTRELIQE
VADEAIGTRQLPLPDGSVYDIDGEWATIQMYPSLSVALGEEITPQTTVDR
LRGIADSLGLEKDPAIHDNRGFGHGKLIEELWERTVGKSLSAPTFVKDFP
VQTTPLTRQHRSIPGVTEKWDLYLRGIELATGYSELSDPVVQRERFADQA
RAAAAGDDEAMVLDEDFLAALEYGMPPCTGTGMGIDRLLMSLTGLSIRET
VLFPIVRPHSN*
```

The plasmid was transformed into BL21 (DE3) cells (Stratagene) using the heat shock methods prior to plating onto LB agar plates supplemented with 50 ug⁻¹ mL⁻¹ ampicillin and incubated overnight at 37° C. Cell scrapings were taken and used to inoculate Autoinduction media (Studier, 2005) supplemented with 50 ug⁻¹ mL⁻¹ ampicillin. Cultures were grown at 20° C. 200 rpm shaking for forty-eight hours to allow expression of the protein. Cultures were pelleted at 3,500 g, 4° C. for 30 minutes prior to being frozen at −20° C. until required. Cells were defrosted and resuspended in buffer A (100 mM HEPES, 150 mM NaCl, 20 mM imidazole, pH 7.5) supplemented with 10 ug⁻¹ mL⁻¹ DNAse (Sigma) and protease inhibitor tablets (Pierce) prior to being lysed at 30 KPSI on a Constant Cell Disruption System (Constant Systems, UK). Cell debris was removed by centrifugation at 40,000 g 4° C. for 30 minutes prior to the supernatant being filtered to GFA prefilter. Supernatant was incubated with 5 mL Ni Sepharose HP (GE healthcare) equilibrated in buffer A for 1.5 hrs. An initial wash step was carried out to remove His Rich proteins over 4 column volumes against 5% buffer B (100 mM HEPES, 150 mM NaCl, 500 mM imidazole, 5% Glycerol pH 7.5). The protein was then eluted in 2×2 CV Buffer B. Fractions containing His-TEV-MtKRS were analyzed by SDS-PAGE. The protein was subjected to dialysis overnight with buffer C (100 mM HEPES, 150 mM NaCl, 5% Glycerol, pH 7.5). The samples were then subjected to gel filtration using a calibrated Superdex 200 50/60 column (GE healthcare) equilibrated with buffer C. MtKRS eluted as a dimer. Samples were subjected to ESI-TOF analysis by the University of Dundee Proteomics Facility to further confirm their identity. Typically, the final samples were in excess of 80% pure by SDS-PAGE analysis.

Mycobacterium tuberculosis Lysine tRNA Synthetase Biochemical Assay Methodology (MtKRS).

Briefly MtKRS, is a dimeric multistep enzyme catalysing the reaction of L-lysine and ATP in the first step and in the second step the transfer of lysine onto the respective tRNA. In the first step pyrophosphate is released as a by-product and it is this that is monitored. MtKRS enzyme activity was tested in a BioMol Green based endpoint assay. A pyrophosphatase enzyme is continuously driving the first step of the reaction by hydrolysing the released inorganic pyrophosphate (PPᵢ) creating free phosphate (Pᵢ). The Pᵢ is detected using Biomol Green reagent (ENZO®) which changes colour from orange to green and is monitored by measuring absorbance at 650 nm.

Test compounds were solubilized in DMSO at a top concentration of 10 mM and serially diluted 1 in 3 to achieve a range of final assay concentrations of 50 μM to 2.5 nM. Compound stocks were pre-plated (250 nL) into 384-well plates (Greiner 781101). The assay mixture was added to the compounds with a reaction volume of 50 μL per well. The final assay mixture contained 40 nM MtKRS, 30 mM Tris-HCl pH8, 40 mM MgCl₂, 140 mM NaCl, 30 mM KCl, 0.01% Brij-35, 1 mM DTT, 30 μM ATP, 12 μM L-lysine, 0.5 U/mL yeast pyrophosphatase (Sigma). After 20 hours of incubation, in a vapour chamber at room temperature, the reaction was stopped by addition of an equal amount of BioMol Green (ENZO®). The stopped reaction mix was incubated for 20 minutes and absorbance read at 650 nm on BMG PHERAstar® FSX plate reader (BMG Labtech if Cary, NC, USA).

Onto each plate 100% and 0% inhibition controls were included. For 0% control DMSO was added instead of compound. 100% inhibition controls lacked L-lysine. Data were analysed by calculating the percentage inhibition compared to the maximum (100%) and minimum (0%) assay controls. Concentration effect curves were fitted using nonlinear regression within ActivityBase and IC₅₀ values were determined using standard protocols.

The results of Assay 1 are provided in Table 1.

Assay 2 Mycobacterium tuberculosis In Vitro H37Rv Extracellular Inhibition Assay (OD Readout)

Mtb strain was cultured in Middlebrook 7H9 medium supplied with 10% ADC and 0,025% Tyloxapol, then incubated at 37° C. for approximately 10 days. Following a purity check, subculture was performed in Middlebrook 7H9 medium supplied with 10% ADC and 0,025% Tyloxapol up to OD (600 nm)=0.01 and incubated at 37° C. 4-6 days. By measuring the OD, the inoculum was adjusted to OD (600 nm)=0.00125 that is equivalent to 1×10⁵ cfu/mL.

50 ul of the inoculum was dispensed per well in every assay plate (384 plates). Plates were placed in a sealed box to prevent evaporation and incubated at 37° C. for 8 days. Then the lids were removed, and the plates were covered a seal. Plates were then read by Abs 590 nm in Envision. If the window between negative control with Rifampicin (Row 18) and positive control (Row 6) was not greater than or equal to 3 times, plates were incubated at 37° C. for one or two additional days and read again. The results of Assay 2 are provided in Table 1.

Assay 3 Mycobacterium tuberculosis In Vitro H37Rv in Human Macrophages THP-1 Inhibition Assay

### (Intracellular Assay)

Intracellular screening is a valuable tool for identifying new antituberculosis compounds that are active in human macrophages. This ex-vivo assay may represent physiological conditions that mimic disease and take into consideration the favourable contribution of host cells. (Sorrentino, F. et al. (2016) Antimicrob. Agents Chemother. 60 (1), 640-645.)

Procedure was carried out as described in Sorrentino, F. et al. (2016) Antimicrob. Agents Chemother. 60 (1), 640-645 (supplemental material), except that before THP-1 infected cells were seeded in 384 well plates, infected macrophages were filtered in the last step of wash steps with a 40 μm cell strainer to remove cell clumps and obtain single cell suspension.

The results of Assay 3 are provided in Table 1.

Assay 4 Acute Mycobacterium tuberculosis In Vivo Assay

Specific pathogen-free, 8-10 week-old female C57BL/6 mice were purchased from Envigo Laboratories and were allowed to acclimate for one week.

Mice were intratracheally infected with approximately 100.000 CFU/mouse (Mycobacterium tuberculosis H37Rv). Moxifloxacin was used as an interassay control. Example 1 and Moxifloxacin were administered once a day via oral from day 1 to day 8 after infection, both included. Moxifloxacin was administered at 30 mg/kg in Captisol 20% and Compounds 45-54 were administered in 1% Methylcellulose at doses from 1 to 300 mg/kg. The volume of administration was 20 mL/kg. Mice could eat and drink ad libitum. Lungs were harvested on day 9 after infection. All lung lobes were aseptically removed, homogenized and frozen. Homogenates were unfrozen and plated in 10% OADC-Middlebrook7H11 medium+0.4% activated charcoal for 18 days at 37° C.

Blood samples were obtained at different time points from infected mice to measure the levels of the tested compounds. Animals were euthanized by CO₂.

All animal studies were ethically reviewed and carried out in accordance with European Directive 2010/63/EU and the GSK Policy on the Care, Welfare and Treatment of Animals.

The results of Assay 4 are provided in Table 1.

### Biological Activity Conclusion

In light of the results of the above-described MtKRS enzyme assay, extracellular assay 2, intracellular assay 3 and in vivo assay 4, it is concluded that Compounds 45-54 exhibit good activity against both Mycobacterium tuberculosis lysine tRNA synthetase and Mycobacterium tuberculosis growth.

TABLE 1
Biological Data

| | Assay 1 | Assay 2 | Assay 3 | Assay 4 | Log PCR reduction |
|---|---|---|---|---|---|
| | Mtb KRS | MIC (μM) | MIC (μM) | ED99 | in acute efficacy |
| Example | (μM) | extracellular | intracellular | (mg/kg) | study @ 50 mg/kg |
| 45 | 0.8 | 1 | 0.8 | 49 | 1.8 |
| 46 | 0.18 | 0.5 | 1.1 | 7 | 3 |
| 47 | 0.4 | 0.9 | 0.6 | ND | 2.1 |
| 48 | 0.6 | 0.8 | 0.6 | ND | 1.9 |
| 49 | 0.3 | 0.2 | 0.2 | ND | 2.6 |
| 50 | 0.05 | 0.07 | 0.07 | 12 | 2.5 |
| 51 | 0.2 | 1 | 0.3 | ND | ND |
| 52 | 0.08 | 0.1 | 0.06 | ND | 0.4 (CFU reduction |
| | | | | | @12 mg/kg) |
| 53 | 0.3 | ND | 0.3 | ND | ND |
| 54 | 0.2 | ND | ND | ND | ND |

ND = not determined

1. A compound of Formula (I): 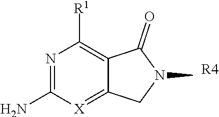
or a pharmaceutically acceptable salt thereof, wherein
R⁴ is 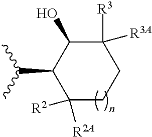
R₁ is methoxy, ethoxy, pyrazolyl, substituted pyrazolyl or —NHMe;
R² and R^{2A} are each independently hydrogen or fluorine;
R³ and R^{3A} are each independently hydrogen or methyl;
X is CH or N; and
n is 1 or 2.

2. The compound or pharmaceutically acceptable salt thereof according to claim 1, wherein n is 1.

3. The compound or pharmaceutically acceptable salt thereof according to claim 1, wherein n is 2.

4. The compound or pharmaceutically acceptable salt thereof according to claim 1, wherein R³ and R^{3A} are each hydrogen.

5. The compound or pharmaceutically acceptable salt thereof according to claim 1, wherein R² and R^{2A} are each fluorine.

6. The compound or pharmaceutically acceptable salt thereof according to claim 1, wherein R¹ is ethoxy.

7. The compound or pharmaceutically acceptable salt thereof according to claim 6, wherein the compound is selected from the group consisting of:

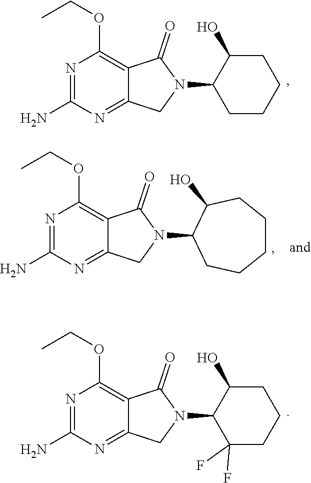

8. The compound or pharmaceutically acceptable salt thereof according to claim 1, wherein R¹ is methoxy.

9. The compound or pharmaceutically acceptable salt thereof according to claim 8, wherein the compound is selected from the group consisting of:

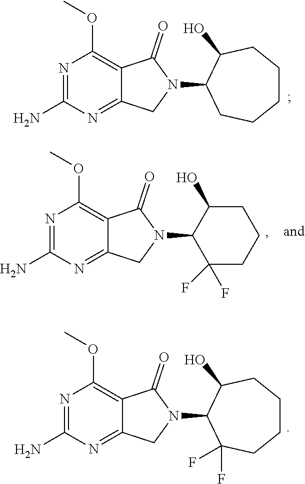

10. The compound or pharmaceutically acceptable salt thereof according to claim 1, wherein R¹ is pyrazolyl or 4-ethoxy-1H-pyrazol-1-yl.

11. The compound or pharmaceutically acceptable salt thereof according to claim 10, wherein the compound is selected from the group consisting of:

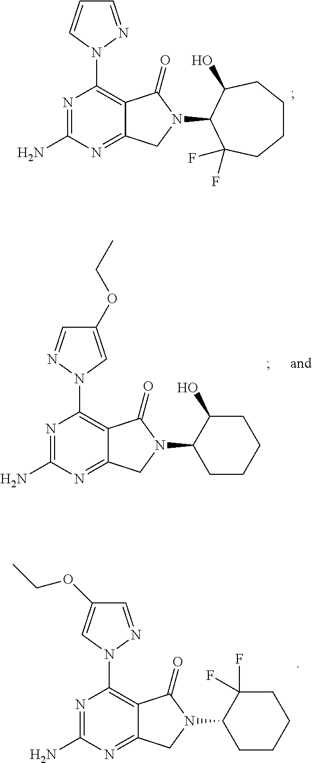 [SMILES: Nc1nc2c(c(-n3cccn3)n1)C(=O)N([C@H]1[C@@H](O)CCCCC1(F)F)C2 ; CCOc1cnn(-c2nc(N)nc3c2C(=O)N([C@@H]2CCCC[C@@H]2O)C3)c1 ; CCOc1cnn(-c2nc(N)nc3c2C(=O)N([C@H]2CCCCC2(F)F)C3)c1]

12. The compound or pharmaceutically acceptable salt thereof according to claim 1, wherein R¹ is —NHMe.

13. The compound or pharmaceutically acceptable salt thereof according to claim 12, wherein the compound is

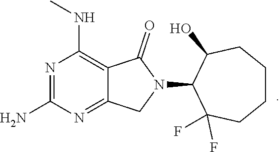 [SMILES: CNc1nc(N)nc2c1C(=O)N([C@H]1[C@@H](O)CCCCC1(F)F)C2]

14. A compound selected from the group consisting of:
2-amino-4-ethoxy-6-[(1R,2S)-2-hydroxycyclohexyl]-7H-pyrrolo[3,4-d]pyrimidin-5-one;
2-amino-4-ethoxy-6-((1R,2S)-2-hydroxycycloheptyl)-6,7-dihydro-5H-pyrrolo[3,4-d]pyrimidin-5-one;
2-amino-6-((1S,6S)-2,2-difluoro-6-hydroxycyclohexyl)-4-ethoxy-6,7-dihydro-5H-pyrrolo[3,4-d]pyrimidin-5-one;
2-amino-6-((1R,2S)-2-hydroxycycloheptyl)-4-methoxy-6,7-dihydro-5H-pyrrolo[3,4-d]pyrimidin-5-one;
2-amino-6-((1S,6S)-2,2-difluoro-6-hydroxycyclohexyl)-4-methoxy-6,7-dihydro-5H-pyrrolo[3,4-d]pyrimidin-5-one;
2-amino-6-((1S,7S)-2,2-difluoro-7-hydroxycycloheptyl)-4-methoxy-6,7-dihydro-5H-pyrrolo[3,4-d]pyrimidin-5-one;
2-amino-6-((1S,7S)-2,2-difluoro-7-hydroxycycloheptyl)-4-(methylamino)-6,7-dihydro-5H-pyrrolo[3,4-d]pyrimidin-5-one;
2-amino-6-((1S,7S)-2,2-difluoro-7-hydroxycycloheptyl)-4-(1H-pyrazol-1-yl)-6,7-dihydro-5H-pyrrolo[3,4-d]pyrimidin-5-one;
2-amino-6-((1S,2R)-2-hydroxycycloheptyl)-4-methoxy-6,7-dihydro-5H-pyrrolo[3,4-d]pyrimidin-5-one;
2-amino-4-ethoxy-6-((1S,2R)-2-hydroxycycloheptyl)-6,7-dihydro-5H-pyrrolo[3,4-d]pyrimidin-5-one;
2-amino-4-(4-ethoxy-1H-pyrazol-1-yl)-6-((1R,2S)-2-hydroxycyclohexyl)-6,7-dihydro-5H-pyrrolo[3,4-d]pyrimidin-5-one; and
(S)-2-amino-6-(2,2-difluorocyclohexyl)-4-(4-ethoxy-1H-pyrazol-1-yl)-6,7-dihydro-5H-pyrrolo[3,4-d]pyrimidin-5-one.

15. A compound which is 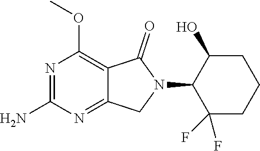 [SMILES: COc1nc(N)nc2c1C(=O)N([C@H]1[C@@H](O)CCCC1(F)F)C2]
or a pharmaceutically acceptable salt thereof.

16. A pharmaceutical composition comprising (a) the compound or pharmaceutically acceptable salt thereof according to claim 1, and (b) a pharmaceutically acceptable excipient.

17. A kit comprising the compound or a pharmaceutically acceptable salt thereof according to claim 1, and instructions for administering to a human in need thereof.

18. A method of treating a mycobacterial infection in a human in need thereof, the method comprising administering to said human a therapeutically effective amount of a compound or pharmaceutically acceptable salt thereof as defined in of claim 1.

19. A method of treating a disease caused by infection with a mycobacterium in a human in need thereof, the method comprising administering to said human a therapeutically effective amount of a compound or a pharmaceutically acceptable salt thereof as defined in claim 1.

20. (canceled)

21. (canceled)

22. The method according to claim 18, wherein the mycobacterial infection is a Mycobacterium tuberculosis infection.

23. (canceled)

24. The method according to claim 19, wherein the disease is tuberculosis.

25. (canceled)

26. A combination of (a) a compound or pharmaceutically acceptable according to claim 1; and (b) at least one other anti-mycobacterial agent.

27. The combination according to claim 26, wherein the at least one other anti-mycobacterial agent is an anti-tuberculosis agent.

28. The combination according to claim 27, wherein the anti-tuberculosis agent is selected from the group consisting of: isoniazid, rifampin, pyrazinamide, ethambutol, rifapentine, clofazimine, ethionamide, prothionamide, isoxyl, thiacetazone, rifabutin, 4-aminosalicylic acid, cycloserine, spectinamide 1810, a fluoroquinolone such as moxifloxacin, gatifloxacin or levofloxacin; a diarylquinoline such as bedaquiline or TBAJ-587 or TBAJ-876; nitroimidazo-oxazine PA-824, delamanid, an oxazolidinone such as linezolid, tedizolid, radezolid, sutezolid (PNU-100480), posizolid, Delpazolid or TBI-223; SPR720, EMB analogue SQ109, OPC-167832, telacebec, spectinamide 1810, GSK3036656, GSK2556286, GSK3211830, GSK3778839, GSK3729098, GSK3653038, a benzothiazinone such as BTZ043 or macozinone; an azaindole such as TBA-7371, a dihyrdocarbostyril derivative such as OPC-167832; a dinitrobenzamide, a beta-lactam such as meropenem, faropenem, ertapenem, tebipenem, sanfetrinem; a beta-lactam combination such as amoxicillin-clavulanate, or an aminoglycoside such as kanamycin, amikacin, capreomycin or streptomycin.

29. The combination according to claim 27, wherein the anti-tuberculosis agent is selected from the group consisting of: isoniazid, rifampin, pyrazinamide, ethambutol, moxifloxacin, rifapentine, clofazimine, ethionamide, prothionamide, isoxyl, thiacetazone, bedaquiline, TBAJ-587, nitroimidazo-oxazine PA-824, delamanid, linezolid, tedizolid, radezolid, sutezolid, posizolid, TBI-223, EMB analogue SQ109, OPC-167832, GSK3036656, GSK2556286, GSK3211830, BTZ043, PBTZ169, TBA-7371, a dinitrobenzamide, a beta-lactam, meropenem, faropenem, ertapenem, tebipenem, beta-lactam combinations, and amoxicillin-clavulanate.

30. The combination according to claim 26-, further comprising an antiviral agent.

31. The combination according to claim 30, wherein the antiviral agent is an antiretroviral agent selected from the group consisting of: abacavir, atazanavir, bictegravir, cabotegravir, darunavir, delavirdine, didanosine, dideoxyinosine, dolutegravir, doravirine, efavirenz, elvitegravir, emtricitabine, etavirine, fosamprenavir, fostemsavir, indinavir, slatravir, lamivudine, lopinavir, maraviroc, nelfinavir, nevirapine, raltegravir, rilpiverine, ritonavir, saquinavir, stavudine, tipranavir, tenofovir, tenofovir alafenamide, tenofovir disoproxil fumarate, zalcitabine, and zidovudine.


SEQUENCE LISTING (3 sequences)

SEQ ID NO: 1 - protein, 13 aa, synthetic construct
  source 1..13: note = His Tag
MGSSHHHHHHGSS

SEQ ID NO: 2 - protein, 9 aa, synthetic construct
  source 1..9: note = TEV Clevage site
GENLYFQGH

SEQ ID NO: 3 - protein, 505 aa, Mycobacterium tuberculosis
  source 1..505: note = MtKRS
MSAADTAEDLPEQFRIRRDKRARLLAQGRDPYPVAVPRTHTLAEVRAAHPDLPIDTATEDIVGVAGRVIFARNSGKLCFATLQDGDGTQLQVMISLDKVGQAALDAWKADVDLGDIVYVHGAVISSRRGELSVLADCWRIAAKSLRPLPVAHKEMSEESRVRQRYVDLIVRPEARAVARLRIAVVRAIRTALQRRGFLEVETPVLQTLAGGAAARPFATHSNALDIDLYLRIAPELFLKRCIVGGFDKVFELNRVFRNEGADSTHSPEFSMLETYQTYGTYDDSAVVTRELIQEVADEAIGTRQLPLPDGSVYDIDGEWATIQMYPSLSVALGEEITPQTTVDRLRGIADSLGLEKDPAIHDNRGFGHGKLIEELWERTVGKSLSAPTFVKDFPVQTTPLTRQHRSIPGVTEKWDLYLRGIELATGYSELSDPVVQRERFADQARAAAAGDDEAMVLDEDFLAALEYGMPPCTGTGMGIDRLLMSLTGLSIRETVLFPIVRPHSN